(19) United States
(12) Patent Application Publication
(10) Pub. No.: US 2012/0040337 A1
(43) Pub. Date: Feb. 16, 2012

(54) Conformational Epitope Initiated Signal Amplification

(76) Inventors: John T. Unger, Norwood, MA (US); Rolf Hilfiker, Allschwil (CH)

(21) Appl. No.: 13/263,367

(22) PCT Filed: Apr. 9, 2010
(86) PCT No.: PCT/US2010/030505

§ 371 Date: Oct. 7, 2011

Related U.S. Application Data
(60) Provisional application No. 61/168,299, filed on Apr. 10, 2009.

(51) Int. Cl. G01N 21/64; C12Q 1/70; G01N 21/75

(52) U.S. Cl. 435/5; 436/501; 435/7.1

(57) ABSTRACT

This invention relates to a method to generate a signal used to detect the presence or quantity of a biomarker in a sample. The signal generating reaction is initiated when the biomarker under assay interacts with a specific binding partner. The interaction produces a structural change in the binding partner that is recognized by additional binding partners capable of generating a signal. The reaction produces a localized cluster of signaling molecules that can be detected above background. The signaling cluster is detectable within minutes when interrogated in a chamber of specific dimensions. The presence of the signaling clusters is a qualitative indication of the presence of the analyte, while the number of signaling clusters detected is a direct quantification of the number of biomarker molecules in the sample. The reaction can be formatted to detect proteins, nucleic acids, cells or other informative biomarkers.

### RELATED APPLICATIONS

This application claims priority to U.S. Provisional Patent Application Ser. No. 61/168,299 filed Apr. 10, 2009, which is incorporated in its entirety herein.

### FIELD OF INVENTION

The present technology is in the general field of immunochemistry (immunoassays), nucleic acid chemistry (nucleic acid assays), pathogen identification and cellular interrogation. The present technology is a method of generating a signal to identify the presence or quantity of an analyte in a sample. The signal generating reaction is initiated when, in a first step, a primary binder reacts to its specific target, and this first step produces a conformational change in the primary binder that exposes one or possibly more than one hidden epitope on the primary binder. In a second step, the exposed epitope is used as a binding site for a secondary binder that can generate a signal. The reaction forms a localized cluster of molecules that produces a signal above background with time when detection takes place in a chamber of specific dimensions using an appropriate optical system.

### BACKGROUND OF THE INVENTION

There is no clearly definable lower limit of assay sensitivity for clinical diagnostics. Assay sensitivity translates into detecting fewer copies of informative biomarkers, which translates into earlier detection of disease, which translates into more effective treatment of disease. At the present time, there are many significant unmet medical needs that can be addressed by a rapid, ultra high sensitivity technology. The early detection of cancers and the rapid detection of low levels of viruses, fungi, bacteria and agents of terror are notable examples. In addition, assay sensitivity translates into a smaller sample volume needed to perform an assay, which is of particular benefit when caring for pediatric and geriatric patients. Assay sensitivity also translates into rapid time to results which enables simple and cost effective instrumentation to be developed to automate the technology processing steps.

At the present time, the methods used to detect ultra low levels of a biomarker fall into one of four categories: target amplification, which increases the target copy number to a detectable level; signal amplification, which increases the number of analyte specific detection molecules to a statistically significant level above background; a combination of both; or the biomarker appears in multiple copies on the entity being interrogated.

There are many signal amplification strategies. The most commonly used strategy utilizes a primary binder, the molecule that recognizes the analyte under assay, to carry multiple signaling molecules, such as a radioactive isotope, a fluorescent molecule or an enzyme that is used to catalyze the formation of a signal generating product. More complicated strategies tend to build multiple layers around the primary binder which increase the number of signal generating molecules associated with the primary binder. Another approach packages a large number of signal generating molecules into a liposome that has multiple copies of a binder on its surface. Others have combined target amplification and immunoassay by developing antibody-nucleic acid conjugates, or constructed conjugates of binders and nano-particles with large signal output. In cases where multiple copies of the biomarker appear on the entity being interrogated, this endogenous amplification can be used to generate enhanced signal.

All the techniques outlined above have been successfully employed to increase signal. Nonetheless, an easy-to-use, low cost, rapid, ultra sensitive and specific detection system has not been developed. Although it is reasonable to assume that rapid high sensitivity results are desirable in most applications, it is also possible that there will be applications of the methods described herein where neither time to result nor sensitivity is critical. The methods described herein need not be limited to rapid, high sensitivity applications.

### BRIEF SUMMARY OF THE INVENTION

The techniques known in the art to measure low levels of a marker are ultimately limited by the dissociation rate constants of the specific binding partners, the amount of detection signal generated and the non specific binding of the conjugate that carries the signal generating molecules. Non specific binding is a major contributor to background noise which ultimately determines the limit of sensitivity or minimum detectable level of the analyte under assay.

The present technology provides a method of generating a signal used to detect or quantify an analyte in a sample, specifically ultra low levels of the analyte, for example, an informative biomarker.

The signal generating reaction of the present technology is initiated when, in a first step, a primary binder binds to its specific target, and this first step produces an analyte-primary binder complex which produces a conformational change in the primary binder that exposes one or possibly more than one hidden epitopes on the primary binder. In a second step, the newly exposed epitope or epitopes are used as a binding site for a secondary binder that can generate a signal.

Numerous examples of systems that can be used for generating the signal of the present technology are described herein. However, the invention is not limited to those examples, and those with expertise in this area will be aware of other alternatives. The various exemplary systems described in the present technology use a primary binder which has one or more epitopes that are hidden before binding to the analyte of interest and which become exposed after binding to the analyte of interest. These newly exposed epitopes are known as conformational epitopes. The one or more newly exposed epitopes on the primary binder then become the binding site or sites for at least one secondary binder, which is a component of the signal-generating system. The various formats of the present technology produce a cluster of signaling molecules based on conformational epitopes. These clusters are detectable when placed in a thin chamber, excited with a laser or other light or energy source. The emitted light is imaged onto a charge coupled device, CCD, or other suitable imaging device. The technology can be summarized as Single Molecule or Single Entity Digital Imaging.

In one aspect, the signal generating system is one in which the secondary binder, also know as a labeled amplifier binder, is designed to not only recognize a conformational epitope on the primary binder and carry a label, but also undergo a conformational change upon binding to the analyte-primary binder complex that produces two or more identical or different epitopes on the secondary binder that can be recognized by additional amplifier molecules that also can produce two or more identical or different epitopes. This reaction, also known as the amplification reaction, is self perpetuating and proceeds without further intervention. The product of the amplification reaction is a cluster or aggregate of molecules. The rate of growth of the cluster at any point in time is a function of amplifier concentration, the amplifier association rate constant, diffusion and length of incubation. The aggregate's radius and signal intensity increases as the amplification reaction proceeds. With time, the signal produced by the aggregate becomes greater than random background noise when detection takes place in a chamber of specific dimensions using an appropriate optical system which includes, for example, a high resolution charge coupled device (CCD) camera, filter, lens and laser. Detection of signal qualitatively identifies the presence of the analyte under assay. The number of aggregates directly quantifies the number of analyte molecules in the sample.

In another aspect of the present technology, the signal generating system is one in which the secondary binder, also known as a fluorescence resonance energy transfer, FRET, labeled Donor (D) or Acceptor (A) binder, is designed to not only recognize a conformational epitope on the primary binder and carry a D or A molecule but also participate in a FRET reaction with a second binder, also know as the FRET partner that is designed to bind to either a stable or conformational epitope that is <10 nm (closer than the Forster distance of the corresponding D and A) away and carry the second member of the FRET signal generating system (i.e., an A or D molecule). The second conformational epitope, recognized by the FRET partner, can be one that (1) was initially hidden but becomes exposed after the primary binder reacts with the analyte, or (2) was exposed before the primary binder reacts with the analyte. The secondary binder may act as either the Donor or Acceptor in FRET signal generation or the second binder FRET partner may act as the other reactant in the FRET signal generation (i.e., either the Donor or Acceptor). The FRET reaction is initiated when the Donor molecule is excited with light at a specific wavelength. The excited donor molecule transfers energy to the acceptor molecule which excites the acceptor molecule which emits light at a specific wavelength that is detected by a photon counting detector. When multiple primary binder molecules are bound to the entity under assay, for example a cell or bacterium, the reaction forms a localized cluster of signaling molecules. The signal generated at any point in time is a function of the number of primary binder binding sites on the entity under assay; the concentration of the primary binder, the FRET labeled Donor or Acceptor and FRET partner; the association rate constant of the primary binder, FRET labeled Donor or Acceptor and FRET partner; the spacing of the FRET labeled Donor or Acceptor and FRET partner; the efficiency of the FRET reaction and the optics of the detection system. With time, the signal produced by the aggregate becomes greater than random background noise when detection takes place in a chamber of specific dimensions using an appropriate optical system which includes, for example, a high resolution CCD camera, filter, lens and laser. Detection of signal qualitatively identifies the presence of the analyte under assay. The number of aggregates directly quantifies the number of analyte molecules in the sample.

In yet another aspect of the present technology, the secondary binder is designed to bind the conformational epitope on the primary binder and upon binding expose one or more conformational epitopes on the secondary binder. The one or more exposed conformational epitopes on the secondary binder are binding sites for the labeled amplifier binder as described above. The secondary binder is acting as a linker between the conformational epitope on the primary binder and the amplification reaction.

In yet a further aspect of the present technology, the secondary binder is designed to bind the conformational epitope on the primary binder and upon binding expose one or more conformational epitopes that are binding sites for the D/A reaction described above for FRET. The secondary binder is acting as a linker between the conformational epitope on the primary binder and the binding sites for the D/A reaction.

In yet another aspect of the present technology, the secondary binder is designed to bind a conformational epitope on the primary binder and upon binding expose one or more conformational epitopes on the secondary binder that are binding sites for the D/A reaction described above and one additional conformational site that is a binding site for an additional secondary binder molecule. The binding of the additional secondary binder exposes one or more conformational epitopes on the additional secondary binder that are binding sites for the D/A reaction described above and one additional conformational site that is a binding site for still another additional secondary binder molecule. The process continues without intervention. The secondary binder is acting as a linear amplifier for the FRET reaction.

In some aspects, the present technology may employ a number of different types of binders, (e.g., primary binders, secondary binders, linkers or amplifiers) that are suitable to produce the desired result. They include, but are not limited to, for example, antibodies, antibody fragments, engineered antibody molecules, interacting proteins and peptides, and nucleic acids.

In further aspects, the present technology may employ antibody and peptide expression libraries and other sources that contain molecules that possess the characteristics suitable for functioning as primary binders, secondary binders, linkers or amplifiers. They include, but are not limited to, for example, phage display libraries, yeast display libraries, ribosome display libraries, hybridomas and aptamer libraries.

Further aspects of the present technology include methods to identify and isolate naturally occurring conformational change epitopes from the expression libraries cited above.

In yet another aspect, the present technology includes the insertion of peptides into primary binders, secondary binders, linkers and amplifiers to produce conformational change epitopes.

In further aspects, the present technology includes the conjugation of molecules to primary binders, secondary binders, linkers and amplifiers to produce conformational epitopes.

In yet further aspects, the present technology include the use of associated molecules that can be used to hide naturally occurring epitopes on primary binders, secondary binders, linkers and amplifiers.

In still further aspects, the present technology may employ biological performance specifications, for example, affinity and specificity, for the primary binder, secondary binders, linkers and amplifiers used in the FRET format.

In yet another aspect of the present technology, certain reaction conditions and performance specifications of the reagents used in the FRET format are described that may be used to obtain the desired signal above background. The specifications include association rate constant of the primary binder, secondary binders and linkers, reagent concentrations, diffusion rate and time of incubation.

In a further aspect of the present technology, certain reaction conditions and performance specifications of the amplifiers used in the amplification format are described that may be used to obtain the desired signal above background. The specifications include association rate constant of the amplifier, reagent concentrations, diffusion rate and time of incubation.

In yet another aspect, the present technology may employ detection specifications to obtain the desired signal above background ratio. The detection specifications include the dimensions of the detection chamber and CCD pixel number.

Another aspect of the present technology includes methods of conjugation to produce reagents.

A further aspect of the present technology includes a method to perform multiplexed testing.

Yet another aspect of the present technology includes a method to detect cells.

Yet another aspect of the present technology includes a method to detect viruses.

Yet a further aspect of the present technology includes a method of sample preparation.

Yet a still further aspect of the present technology includes a method of protein purification.

In yet a further aspect, the present technology provides a method for determining the presence or quantity of an analyte in a sample, comprising reacting each unit of sample with a primary binder, the primary binder having specificity for the analyte, to form an analyte-primary binder complex, wherein the primary binder comprises one or more hidden epitopes, wherein the hidden epitopes of the analyte-primary binder complex become exposed upon the primary binder binding to the analyte; reacting the analyte-primary binder complex with a signal generating system, wherein the signal generating system binds to the exposed epitopes of the analyte-primary binder-complex to form an analyte-primary binder-signal generating system complex and generating a signal, and determining the presence or quantity of the signal as a means of determining the presence or quantity of the analyte.

### BRIEF DESCRIPTION OF SEVERAL VIEWS OF THE DRAWINGS

FIG. 1A a diagram of Binding Reactions to Produce a FRET Signal with Conformational Epitopes.

FIG. 1B shows a diagram of Amplification Reaction when the Primary Binder and Amplifier have Two Identical Conformational Epitopes.

FIG. 1C shows a diagram of Amplification Reaction when the Primary Binder and Amplifiers have Two Different Conformational Epitopes.

FIG. 2A shows a process for Selection of Primary Binders.

FIG. 2B shows a process for Selection of Secondary Binders.

FIG. 3 shows the process for Engineering of Amplifier Molecules.

FIG. 4A shows a process for Engineering Amplifier Molecules with Two Identical Protein Domains.

FIG. 4B shows a process for Engineering Amplifier Molecules with Two Different Protein Domains.

FIG. 5A shows the Initiation of the Amplification Reaction Utilizing an Existing Mab or Ab.

FIG. 5B shows the Initiation of the FRET Reaction Utilizing an Existing Mab or Ab.

FIG. 6A shows the Initiation of the Amplification Reaction Utilizing the C1Q and the FcgR1 Binding Site.

FIG. 6B shows the Initiation of the FRET Reaction Utilizing the C1Q and the FcgR1 Binding Site.

FIG. 7 shows a Method to Engineering Whole Antibody Amplifiers from a C1Q and a FcgR1 Binding Site Binder.

FIG. 8 shows Growth Rate of an Aggregate vs. Time in the Amplification Format Using Two Different Amplifiers.

FIG. 9 shows Radius of an Aggregate vs. Time in the Amplification Format Using Two Different Amplifiers.

FIG. 10 shows Contrast and Number of Amplifier Molecules per Aggregate vs. Time in the Amplification Format Using Two Different Amplifiers.

FIG. 11A predicts the Time Required to Obtain a Contrast Level of 4 or 9 as a Function of Number of Epitopes in the FRET Format.

FIG. 11B shows the Donor and Acceptor Concentration Required to Obtain the Contrast Shown in FIG. 11A.

FIG. 12 shows a Diagram of an Optical System Used to Detect Reaction Products.

FIG. 13 shows the Reagents Required to Isolate or Concentrate Immune Complexes from a Complex Matrix Using Conformational Epitopes.

### ABBREVIATIONS

The following abbreviations are used throughout the specification:

A—Acceptor—An acceptor is a member of a FRET pair. The acceptor is excited by energy transferred by the donor molecule.

Ab—antibody

aM=attomolar=E-18M

Amp or AMP or amplifier molecule—The amplifier molecule is a secondary binder that binds a conformational epitope on a primary binder which is exposed when the primary binder binds its specific analyte. The amplifier molecule upon binding the epitope on the primary binder undergoes a conformational change that produces two or more identical or different epitopes that are recognized by additional amplifier molecules that also produce two or more identical or different epitopes. An amplifier is labeled with a signal generating molecule.

AUA—analyte under assay

AUA-PB complex—analyte under assay-primary binder complex

AUA-PB-SB complex—analyte under assay-primary binder-secondary binder complex

C=contrast obtained on CCD (see Table 4)

C1Q—a 400 kDa (1 kDa=1000 Da) protein formed from 18 peptide chains in 3 subunits of 6.

CCD—charge coupled device

CDR—complementarity determining regions—In the variable (V) domain of an antigen receptor there are three CDRs (CDR1, CDR2 and CDR3). Since the antigen receptors are typically composed of two polypeptide chains, there is a frequency of about six CDRs for each antigen receptor that can come into contact with the antigen (each heavy and light chain contains three CDRs).
H1—CDR H1
H2—CDR H2
H3—CDR H3
L1—CDR L1
L2—CDR L2
L3—CDR L3

CE—conformational epitope—a binding site that is hidden or does not exist but becomes exposed or created when molecules interact

CH1—constant domain 1 heavy chain

CH2—constant domain 2 heavy chain

CH3—constant domain 3 heavy chain

CL—constant domain light chain

C-myc tag—A myc-tag is a polypeptide protein tag derived from the c-myc gene product that can be added to a protein using recombinant DNA technology

CSF—cerebral spinal fluid

CTC—circulating tumor cell

CUA—cell under assay

D—Donor—One member of a FRET pair. The donor molecule carries a fluorescent label that when excited with light of an appropriate wavelength transfers energy to an acceptor molecule.

EPI—any particular epitope

EUA—entity under assay

Fab—antibody fragment containing a VH, CH1, VL and CL domain

Fab′2—antibody fragment containing 2 Fabs

FcgR1—An Fc receptor is a protein found on the surface of certain cells—including natural killer cells, macrophages, neutrophils, and mast cells—that contribute to the protective functions of the immune system

Flag tag—FLAG octapeptide, is a polypeptide protein tag that can be added to a protein using recombinant DNA technology

FRET—fluorescence resonance energy transfer

IHC—immunohistochemistry

His tag—A polyhistidine-tag is an amino acid motif in proteins that consists of at least six histidine (His) residues, often at the N- or C-terminus of the protein

Mab—monoclonal antibody

mM—millimolar (1E-3 M)

MP—magnetic particle

mpix—mega (1E6) pixels

NE—number of epitopes on an EUA

NSB—non-specific binding

PB—primary binder—a binder that binds the AUA, CUA, EUA

PDL—phage display library

SB—secondary binder—a binder that binds a conformational epitope produced when the primary binder binds its target—AUA, CUA, EUA (under certain conditions secondary binders may bind stable epitopes)

SBA—secondary binder labeled with a fluorescent acceptor

SBD—secondary binder labeled with fluorescent donor

scFv—antibody fragment containing a VH and VL domain held together by a peptide linker

SPDP—A heterobifunctional cross linking agent

TTR—time to result

VhH—Camelid single domain antibody

VH—variable heavy chain

VL—variable light chain

V-NAR—Shark variable domain new antigen receptor antibody

| | | |
|---|---|---|
| α | (1) | a constant relating to the emission intensity of D |
| | | and A |
| c(AMP) | (mol l⁻¹ = M) | concentration of AMP |
| C1 | (1) | constant depending on detection system and assay |
| | | reagents |
| C2 | (1) | constant depending on detection system and assay |
| | | reagents |
| const | (m/s) | constant depending on the geometrical properties of |
| | | AMP. |
| CD | (1) | Contrast for Detection |
| D | (m² s⁻¹) | diffusion coefficient of AMP |
| FRET | (I) | = I_{fluo, close}/I_{fluo. random} |
| enhancement | | |
| I_{background} | (1) | Signal intensity of background |
| I_{fluo, close} | (1) | FRET fluorescence intensity when D and A are close |
| | | together |
| I_{fluo, random} | (1) | FRET fluorescence intensity when D and A are far |
| | | apart |
| I_{signal} | (1) | Signal intensity of aggregate |
| ka, kₐ | (mol⁻¹/s⁻¹ = M⁻¹s⁻¹) | association rate constant |
| kd, k_{d} | (s⁻¹) | dissociation rate constant |
| l | (l) | liter (1000 cm³) |
| λₑₓ | (nm) | wavelength at which the system is excited |
| λₑₘ | (nm) | wavelength at which emission is observed |
| MW | (g mol⁻¹) | molecular weight |
| MW(AMP) | (g mol⁻¹) | molecular weight of AMP |
| N_{A} | (mol⁻¹) | Avogadro's Number |
| N_{aggregate} | (1) | Number of AMP in the aggregate |
| n(AMP) | (1) | number of AMP molecules |
| Npix | (1) | Number of pixels the assay volume is imaged |
| | | on |
| N_{layer} | (1) | number of layers the aggregate is built of |
| r_{aggregate} | (m) | radius of aggregate |
| r(PB) | (m) | radius of PB |
| r(AMP) | (m) | radius of AMP |
| t_{layer} | (s) | time to add an additional layer |
| V(AMP) | (m³) | volume of AMP |
| Vassay | (ul) | Assay volume |
| Φ | (1) | volume fraction of AMP in aggregate |

um = micrometer = E−6 m
uM = micromolar = E−6 M
10\hat{ }5 = E5 = 10⁵

### DETAILED DESCRIPTION OF THE INVENTION

Diagnostic medicine relies on the detection and quantification of proteins, nucleic acids, cells, pathogens, viruses and other biomarkers to evaluate health, identify disease, monitor disease progression and regression and assess therapeutic efficacy or failure. No technology available today integrates the detection of this diverse spectrum of targets onto a single platform that delivers rapid and ultra sensitive results. In addition, diagnostic assays are frequently limited by reagent specificity, non specific interactions and sample processing challenges.

There is a need in the art for a simple, cost effective method to detect ultra low concentrations of an analyte, for example, a protein, a virus, a cell, a nucleic acid, or the like that does not require an enzymatic reaction or amplification of the target molecule. The present technology provides a method of detecting very low levels of an analyte in a sample which will be described herein.

In the method described in the present technology, the binding of the target analyte to its specific primary binder exposes hidden sites in or on the specific primary binder. The newly exposed sites are not capable of producing a signal but act as docking sites for molecules that can generate a signal. The signal generating elements are not quenched but capable of signal generation at all times. Signal generation is dependent upon newly exposed epitopes and the formation of a localized cluster of signaling molecules in solution. The method of the present technology relies only on the target present in the sample. No target amplification or enzymes are required for signal generation. Quantification of target is by spatially resolving cluster not by bulk solution interrogation. In addition, the method described herein is appropriate for protein, cells and nucleic acid detection.

Conformational epitopes offer a new way to deal with ultra sensitive detection, control specificity and isolate or concentrate an analyte or entity under assay. In addition, conformational epitopes offer a way to integrate protein, nucleic acid, cell and pathogen detection on a single platform.

Some embodiments of the present technology use conformational epitopes for the ultra sensitivity detection of analytes, for example, cells, proteins, and viruses. Rather than trying to separate the specific signal from background noise, as most high sensitivity formats do, embodiments of the present technology center on concentrating the specific signal into a small volume in the presence of background noise. The concentration effect produces an aggregate that has a clear signal above background when detection takes place in a chamber of specific dimensions. The binding of a uniquely designed primary binder to the analyte under assay initiates a reaction that leads to the formation of a signal generating cluster or aggregate. The number of clusters formed is a direct quantification of the number of analyte molecules in the sample. The system can be controlled by calibrators that confirm quantification accuracy. The method can be formatted to detect a protein, nucleic acid, cell, virus or any molecule that has an appropriate primary binding partner.

The technology can also be used to qualitatively determine the presence of an analyte in a sample. Although quantitative calibrators are not necessary for this type of assay, a positive and negative control can be incorporated for compliance with good laboratory practices.

Certain embodiments of the present technology are centered on selecting and building binders that can be used as immunoassay or nucleic acid assay reagents. The binders are components of an immunoassay or nucleic acid assay signal amplification system. The signal amplification system is capable of detecting ultra low levels of any biomarker that has a binding partner appropriate for the format.

Certain embodiments of the present technology involve selecting a primary binder. A primary binder is designed or selected to react to its specific target and in so doing produce a analyte-primary binder complex which produces a conformational change in the primary binder that exposes one or possibly more than one hidden epitope on the primary binder, Step 1. The newly exposed epitope or epitopes is used as a binding site for a secondary binder that can generate a signal, Step 2. A primary binder may be selected from, for example, a large protein display or aptamer display library, an existing monoclonal antibody or found in a polyclonal antiserum.

Certain embodiments of the present technology involve selecting a secondary binder. Secondary binders recognize conformational epitopes produced by the primary binder when the primary binder binds its specific target to produce an analyte-primary binder complex. Secondary binders may be directly labeled with a signal generating molecule or act as a linker between the primary binder and signal generation. A secondary binder is selected to meet strict requirements related to specificity, association rate constant and working concentration.

One type of secondary binder, a FRET labeled donor or acceptor binder, recognizes a conformational epitope on the primary binder and carries a Donor or Acceptor molecule. The FRET labeled donor or acceptor binder participates in a FRET reaction with another secondary binder, also know as the FRET partner. The FRET labeled donor or acceptor binder is selected to meet strict requirements related to specificity, association rate constant, working concentration and binding position on the primary binder. In an alternative embodiment, the FRET secondary binder carries neither a Donor nor an Acceptor molecule before it reacts with the primary binder. However, after binding the primary binder, conformational epitopes on the FRET secondary binder become exposed, and these newly exposed epitopes are binding sites for other secondary binders labeled with Donor and Acceptor molecules. In this embodiment, the FRET secondary binder without a donor or acceptor molecule is acting as a linker between the primary binder and the FRET donor and acceptor molecules.

In some embodiments, another type of secondary binder, a FRET partner, binds a stable or conformational epitope on the primary binder that is <10 nm away from the FRET labeled donor or acceptor binder. The FRET partner may bind (1) a conformational epitope that was initially hidden but becomes exposed after the primary binder reacted with the analyte or (2) to a stable epitope, one that was exposed before the primary binder reacted with the analyte. The FRET partner may be labeled with either the Donor or Acceptor molecule. The FRET partner is selected to meet strict requirements related to specificity, association rate constant, working concentration and binding position on the primary binder.

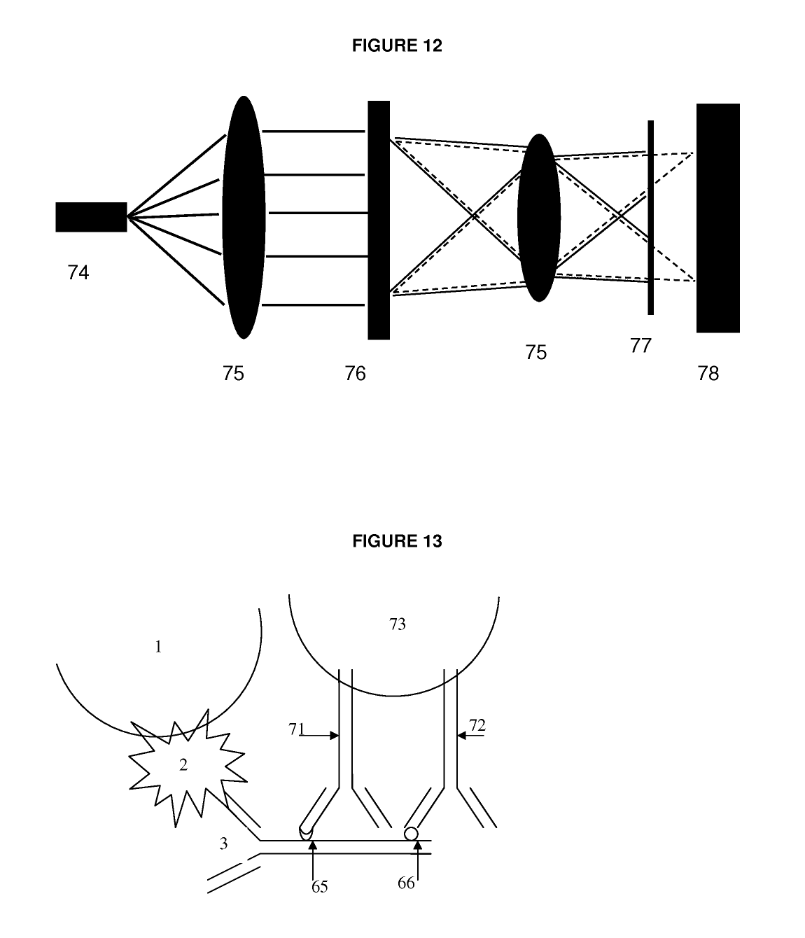

A further embodiment of the technology is the use of a FRET labeled donor or acceptor binder and a labeled FRET partner, together referred to as the FRET pair, to develop a FRET signal to detect the presence of an entity under assay (EUA). The FRET pair must bind to epitopes that are <10 nm apart on the primary binder, FRET linker or FRET amplifier. (See section below for details on FRET linker and FRET amplifier). The FRET pair is labeled with a Donor and Acceptor molecule capable of FRET signal generation. (See section below for details on Donor and Acceptor molecules.) The FRET reaction is initiated when the donor molecule is excited with light at a specific wavelength. The excited donor molecule transfers energy to the acceptor molecule which excites the acceptor molecule which emits light at a specific wavelength that is detected by a photon detector. The signal generated at any point in time is a function of the number of primary binder molecules bound to the entity under assay; the concentration of the primary binder, the FRET labeled D/A binder and the FRET partner; the association rate constant of the primary binder, the FRET labeled D/A binder and the FRET partner, the spacing of the FRET pair, the efficiency of the FRET reaction and the optics of the detection system. With time, the signal produced by the cluster of signaling molecules on the EUA becomes greater than random background noise when detection takes place in a chamber of specific dimensions using an appropriate optical system including, for example, a high resolution CCD camera, filter, lens and laser, see FIG. 12. The major drivers of assay performance are shown in Table 1 which summarizes the time to reach contrast 9 predictions for a FRET cluster as a function of CCD pixel number, donor and acceptor association rate constant, donor and acceptor concentration and number of epitopes displayed on the entity under assay. The data predicts that an aggregate of contrast 9 will be formed in ˜2.5 minutes when the entity displays 10,000 epitopes using a 25 megapixel CCD and a donor and acceptor pair with a ka of 7E5(M⁻¹s⁻¹) at a concentration of 1.2E-8 M.

TABLE 1

| | | Minutes to | Minutes to | Minutes to |
|---|---|---|---|---|
| | Forward | Contrast 9 | Contrast 9 | Contrast 9 |
| CCD | rate | @ 100 | @ 1,000 | @ 10,000 |
| Mega- | ka E5 | epitopes/entity | epitopes/entity | epitopes/entity |
| pixels | (M−1s−1) | (at D/A conc) | (at D/A conc) | (at D/A conc) |
| 5 | 2 | 4430 | 443 | 44 |
| | | (2.3E−11 M) | (2.3E−10 M) | (2.3E−9 M) |
| 10 | 3 | 1470 | 147 | 15 |
| | | (4.6E−11 M) | (4.6E−10 M) | (4.6E−9 M) |
| 15 | 4 | 738 | 74 | 7.4 |
| | | (6.9E−11 M) | (6.9E−10 M) | (6.9E−9 M) |
| 20 | 5 | 441 | 44 | 4.4 |
| | | (9.2E−11 M) | (9.2E−10 M) | (9.2E−9 M) |
| 25 | 7 | 253 | 25 | 2.5 |
| | | (1.2E−10 M) | (1.2E−9 M) | (1.2E−8 M) |
| 30 | 7 | 211 | 21 | 2.1 |
| | | (1.4E−10 M) | (1.4E−9 M) | (1.4E−8 M) |

Constants
200 ul sample volume
Read Chamber (3.2 cm × 3.2 cm × 0.2 mm)
FRET enhancement: 100
c(primary binder) = 50 nM (−> @5E5(M−1s−1), 78% bound in 1 minute)

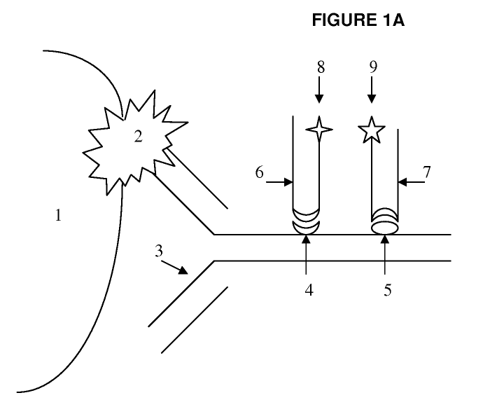

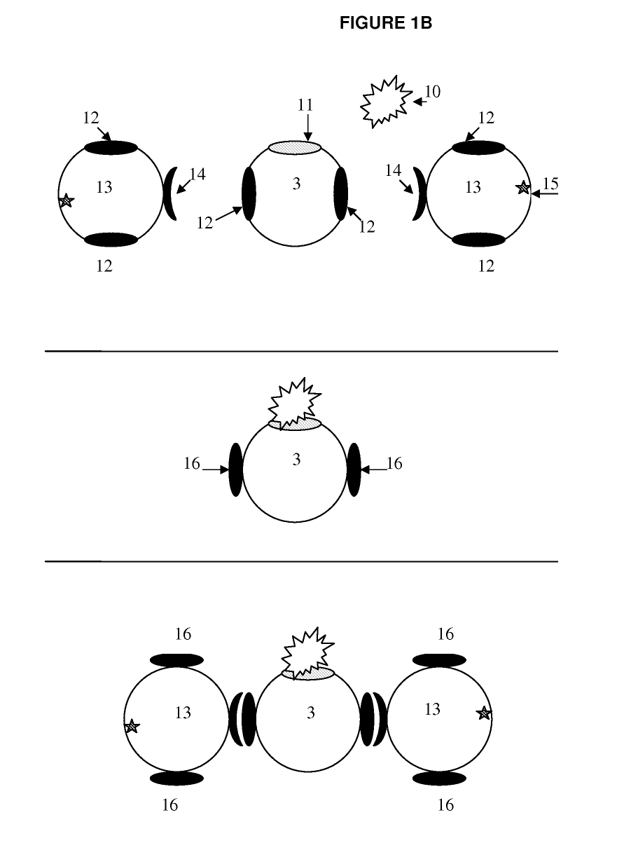

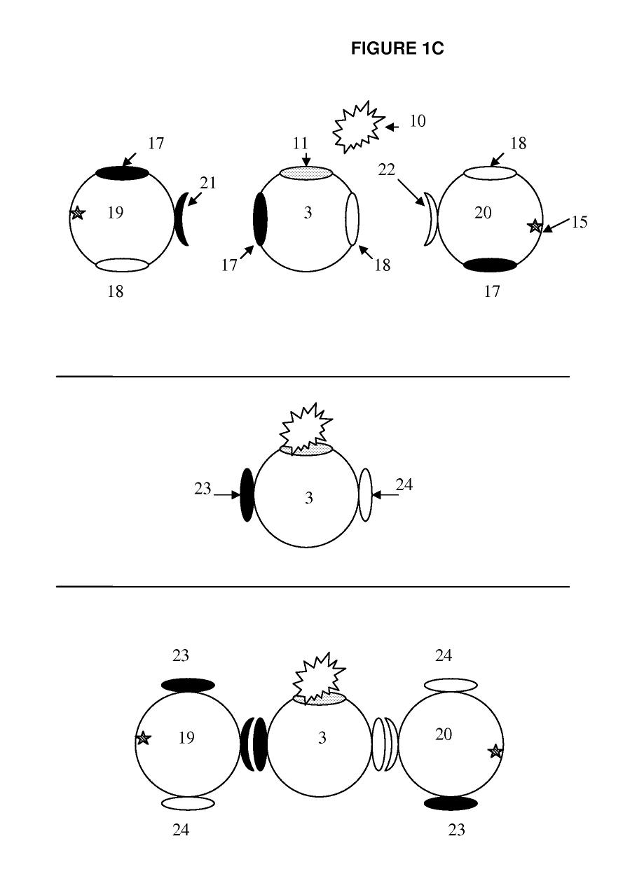

Detection of signal qualitatively identifies the presence of the entity under assay. In addition, the number of aggregates directly quantifies the number of entities in the sample. Calibration of the reagents and instrument can be done at some interval. Calibration includes a no sample control to assess any reagent background and EUA at several levels to assess system performance. FIG. 1A diagrams the alignment of reagents to generate a signal in the FRET format. The entity under assay 1 displays an antigen 2 that is specifically recognized by the primary binder 3. Note the antigen 2 is shown as one copy for the purpose of illustration but may range from <100->100,000 copies on the entity under assay 1. Upon binding to the displayed antigen 2, the primary binder 3 undergoes a conformational change that exposes two epitopes 4, 5 that are within 10 nm of each other or one stable epitope 4 and one conformational epitope 5 that are within 10 nm of each other. The epitopes 4, 5 are binding sites or docking sites for two secondary binders 6, 7. One secondary binder is labeled with a donor molecule 8 and the other secondary binder 9 is labeled with an acceptor molecule. The donor and acceptor molecules are a FRET pair. When the labeled secondary binders 6, 7 are bound to the primary binder epitopes 4, 5, the pair is properly oriented to generate a FRET signal. The solution containing the EUA-primary binder-secondary binders complex is illuminated with a wave length that excites the donor molecule, the excitation energy is transferred to the acceptor molecule which emits a photon at a specific wave length that is detected by an appropriate optical system.

FIG. 12 shows an exemplary optical system that can be used to detect clusters or aggregates produced in the FRET or amplification format. Laser 74 or other suitable light of appropriate wavelength is used to illuminate the detection chamber 76 containing aggregates through a lens 75. Emitted light is collected, filtered through a filter 77 and quantified by a CCD 78 or other position sensitive detector.

In some embodiments, another type of secondary binder, also know as a labeled amplifier binder, is selected or constructed to recognize a conformational epitope on the primary binder or amplifier linker (see section below for details), carry a label and also undergo a conformational change upon binding to the analyte-primary binder complex. The conformational change produces two or more identical or different epitopes that are recognized by additional amplifier molecules that also produce two or more identical or different epitopes. The reaction, also known as the amplification reaction, is self perpetuating and proceeds without further intervention. If some amplifier molecules have only one functional conformational epitope the reaction will still proceed. The labeled amplifier binder may carry a fluorescent molecule or any appropriate signal generating molecule for detection. The product of the amplification reaction is a cluster or aggregate of molecules. The rate of growth of the cluster at any point in time is a function of the amplifier concentration, the amplifier association rate constant, diffusion and length of incubation. The aggregate's radius and signal intensity increases as the amplification reaction proceeds. With time, the signal produced by the aggregate becomes greater than random background noise when detection takes place in a chamber of specific dimensions using an appropriate optical system including, for example, a high resolution CCD camera, filter, lens and laser, e.g., FIG. 12. Amplifier kinetics and the number of pixels on the CCD are major drivers of assay performance. Table 2 summarizes the time to contrast 50 predictions for an amplification cluster as a function of CCD pixel number and amplifier association rate constant. In at least one embodiment, the other constants used in the calculations include 10 uM amplifier concentrations, 200 ul sample volume and a detection chamber of dimensions of about 3.2 cm×about 3.2 cm×about 0.2 mm. The data predicts that an aggregate of contrast 50 will be formed in ˜5 minutes using 2 amplifiers with a ka of 5E5(M⁻¹s⁻¹) and a 20 mega pixel CCD. Detection of signal qualitatively identifies the presence of the analyte under assay. The number of aggregates directly quantifies the number of analyte molecules in the sample. Calibration of the reagents and instrument will be required at some interval. Calibration will include a no sample control to assess any reagent background and AUA at several levels to assess system performance. See FIG. 1B and FIG. 1C.

FIG. 1B diagrams the reagents in the solution followed by the first two steps of the amplification reaction when amplifier molecules have two identical epitopes: the primary binder 3 which has 2 identical hidden epitopes 12 interacts with the analyte (entity) under assay 10, two identical conformational epitopes 16 are exposed; followed by the amplifier molecules 13 interacting with the conformational epitopes 16 on the primary binder 3, which causes the amplifier molecules 13 conformational epitopes 16 to be exposed. 11 is the binding site of the primary binder, 14 is the binding site of the amplifier binder, and 15 is the label on the amplifier binder. The primary binder produced two binding sites and when the amplifiers bind, they produce four binding sites. Additional rounds of amplifier binding produces in principle (see Section “Kinetics of Amplification Reaction”, below) an exponential, (2)̂N, reaction. This diagram represents an amplification reaction when the primary binder and the amplifier each have two identical conformational epitopes. A primary binder with only one conformational epitope can initiate the amplification reaction.

FIG. 1C diagrams the reagents in the solution followed by the first two steps of the amplification reaction when amplifier molecules 19, 20 have two different hidden epitopes 17, 18: the primary binder 3 interacts with the analyte (entity) under assay 10 (where 11 is the binding site of the primary binder) which causes the primary binder's two different hidden conformational epitopes 17, 18 to be exposed 23, 24; followed by two different amplifier molecules 19, 20 (each of which has the same set of two different conformational epitopes 17, 18 but different binding domains 21, 22) interacting with the conformational epitopes 23, 24 on the primary binder 3, which causes their conformational epitopes 23, 24 to be exposed. The primary binder produced two binding sites and when the amplifiers bind, they produce four binding sites. Additional rounds of amplifier binding produces in principle (see Section “Kinetics of Amplification Reaction”, below) an exponential, (2)̂N, reaction. This diagram represents an amplification reaction when the primary binder and the amplifiers each have two different conformational epitopes. In some embodiments, a primary binder with only one conformational epitope can also initiate the amplification reaction.

In some embodiments, another secondary binder, also known as an amplifier linker, is isolated or designed to bind a conformational epitope on the primary binder and upon binding expose one or more conformational epitopes that are binding sites for a labeled amplifier binder. This type of secondary binder acts as a linker between the conformational epitope on the primary binder and the amplification reaction. An amplifier linker is useful when, for example, a labeled amplifier binder has been developed but does not have specificity for a conformational epitope on a primary binder derived from a different source. The amplifier linker would provide the means to utilize the primary binder and the labeled amplifier to generate a signal. The concentration and forward rate constant of the amplifier linker are important to assay performance.

In another embodiment, another secondary binder, also known as a FRET linker, is isolated or designed to bind the conformational epitope on the primary binder and upon binding expose one or more conformational epitopes that are binding sites for a FRET pair. A FRET linker is useful when a FRET pair has been developed but has specificity for conformational epitopes on a primary binder derived from a different source. The FRET linker would provide the means to utilize the primary binder and the FRET pair to generate a signal. The concentration and forward rate constant of the FRET linker are important to assay performance.

In another embodiment, another secondary binder, also known as the FRET linear amplifier, is isolated or designed to bind a conformational epitope on the primary binder and upon binding provide binding sites for a FRET pair and one additional conformational site that is a binding site for an additional FRET amplifier molecule that upon binding provides binding sites for a FRET pair and one additional conformational site that is a binding site for an additional FRET amplifier molecule. In this case, a linear amplification reaction is taking place. The reaction is self sustaining and amplifies the number of FRET pairs associated with the entity (analyte) under assay. The concentration and forward rate constant of the FRET amplifier are important to assay performance.

### Types of Analytes

The amplification format, the FRET format and the FRET linear amplification format described herein can be used to detect a broad range of analytes. The term analyte is intended to mean any molecule in a sample that is being identified or quantified and is interchangeable in the present technology with the entity under assay. An analyte may be displayed on the surface of a cell or virus or be contained within a cell or virus. The analyte or fragments thereof may be present in blood, serum, plasma, other bodily fluids or other sample. Furthermore, the cell or virus itself may be the analyte, since, in working with a phage display library or similar collection of binders, it is sometimes not known which component or components of the analyte serves as the target of the binder.

An analyte can be, but is not limited to, a protein, nucleic acid, lipid, carbohydrate, steroid or other molecule or fragment thereof that can be used to provide information to the analyst (i.e., any informative biomarker). The term protein is intended to mean a chain of amino acids of any sequence, of any length, with any post translational modification to include but not limited to the addition of lipids, carbohydrates, phosphate, acetate or non enzymatic modifications like oxidation. A protein may be present in blood, serum, plasma, bodily fluids or displayed on the surface of a cell or a virus or contained within a cell or a virus. The term nucleic acid is intended to mean a chain of bases of any sequence, of any length, with or without modifications to include, but not limited to, methylation and glycosylation. The nucleic acid under interrogation may exist within a cell or a virus or be found in a bodily fluid or other sample. More specifically, examples of analytes include, but are not limited to, for example, Bacillus anthracis, Variola virus, Francisella tularensis, Yersinia pestis, Ricin toxin, Clostridium botulinum toxin, Staphylococcal enterotoxin B, Clostridium perfringens epsilon toxin, Vibrio cholerae, Candida species, Aspergillus species, Staphylococcus aureus, Staphylococcus epidermidis, HIV, CMV, EBV, HHV6, HHV7, BK, CKMB, Troponin T, Troponin I, Circulating Tumor Cells, Her2, EGFR, CxCR4, Twist1, Ki-67, Mucin 1, Cathepsin D and the like.

A binder used in the present technology can bind an analyte directly or can bind a post synthesis modification made to the analyte.

The term antigen includes a molecule that will produce an immune response which includes the production of antibodies of any class and any molecule that can be used as bait to isolated binders from a phage display, peptide display and aptamer display library. The entity under assay or analyte under assay can be an antigen.

### Types of Binders

The primary binder, secondary binders, linkers and amplifiers used with the present technology can be selected from a list of binders that include, but are not limited to: an antibody; or its binding fragments (a Fab, Fab′2, VH domain, VL domain); or engineered binders, including a scFv, diabody, triabody, tetrabody, minibody; or a shark or camel IgG, or their fragments a VhH or V-NAR, which are disclosed, for example, in Jefferis, Carter, and Holliger, which are incorporated by reference in their entirety. Antibody molecules produced by humans and mice are composed of two heavy and two light amino acid chains. The heavy chain has a variable region and three constant regions CH1, CH2 and CH3. The light chain has a variable region and one constant region, CL. The heavy and light variable regions each participate in forming an antigen binding domain. The constant regions provide structural stability and sites that participate in various immune functions. See, for example, Jefferis.

The primary binder, secondary binders, linkers and amplifiers can be selected from or modeled after other protein binders to include receptor-ligand interactions, peptide-protein, or protein-protein interactions. The primary binder, secondary binders, linkers and amplifiers can also be an aptamer or a nucleic acid sequence. The primary binder, secondary binders, linkers and amplifiers can also be selected from alternative scaffolds including protein A, lipocalin, fibronectin, ankyrin or thioredoxin. See, for example, Skerra et al. The primary binder, secondary binders, linkers and amplifiers may be derived from any species. The primary binder, secondary binders, linkers and amplifiers can be any binding pair that can be selected or designed to modulate an epitope that is used to generate or amplify a signal that is used to detect a biomarker.

### Sources of Binders

In the process of developing antibody therapeutics, the pharmaceutical industry has compiled a significant body of knowledge related to antibody engineering. The half life of a therapeutic or the presence or absence of various functions like antibody-dependent cellular cytotoxicity or complement-dependent cytotoxicity can be modulated depending on the desired outcome. During the evaluation of the drug Rituxan it was discovered that the human and murine C1Q binding sites are different. See, Idusogie et al. There are also many examples of binders to the binding domains of C1Q that compete or inhibit its binding, Gadjeva et al., incorporated by reference in its entirety. In addition, engineered immunoglobulin molecules are commercially available (See, for example, www.invivogen “IgG Fc Engineering”).

Primary binders, secondary binders, linkers and amplifiers can be isolated from a number of sources including, but not limited to, antibody fragment display libraries, peptide display libraries, aptamer display libraries, hybridomas or modeled after protein-protein or peptide-protein interactions. Antibody fragment libraries may contain binders that are not produced or permitted by a functional immune system. In addition, peptide and aptamer display libraries can add even more diversity and complexity for selecting potential binders.

Antibody fragment and peptide display libraries containing billions of members have been prepared and are routinely used to identify binders for pharmaceutical and diagnostic purposes. Libraries displaying antibody and peptide fragments have been expressed on the surface of phage, yeast and ribosomes. Binders are isolated from these libraries by standard panning procedures that include repetitive cycles of binding, washing, release and enrichment. See, for example, Maynard et al, incorporated by reference in its entirety. Libraries are available under a number of business arrangements including: library purchase, fee for service development or partnership relationships.

Libraries of the complexity described above are suitable for the isolation and manipulations used to select the binders employed in embodiments of the present technology. In addition to large diversity of molecular structure, each isolate, in a phage display library for example, contains the nucleic acid sequence of the expressed binder along with sites designed to permit sequence manipulation using standard molecular biology procedures. These libraries are designed to permit a rapid and convenient method to create variance or arrange protein topography as desired. They are ideal for the strategic insertion of epitopes into a binder. Furthermore, the technology to engineer library isolates into whole antibodies or improve the binding affinity of an isolate is well documented.

Binders can also be isolated from hybridomas expressing high affinity antibodies. The hybridoma may produce an antibody that contains a naturally occurring conformational epitope or has an epitope inserted into the antibody or conjugated to the antibody.

In addition, binders can be isolated from an aptamer display library. An isolated aptamer can produce a naturally occurring conformational epitope upon interacting with its binding partner, or an epitope can be inserted into the aptamer or conjugated to the aptamer.

Alternatively, binders can be modeled after known protein-protein or peptide-protein interactions, which will be described in more detail below.

Binders can be derived or isolated from any species, for example but not limited to, humans, mouse, rat, rabbit, and the like. There may be advantages to using a non-human phage display library, for example, a mouse phage display library, for the isolation and development of primary binders, secondary binders, linkers and amplifiers for human clinical applications. If the AMPs are selected from a human scFv or Fab library or these fragments are engineered into complete antibodies and the CEs are from naturally occurring widely used frameworks, non-specific initiation of an amplification center may occur if immune complexes are present in the sample. This problem can be avoided by selecting binders from a mouse library. Human anti-mouse antibodies may be found in some human sera but its potential inhibition should not be significant because reagent concentrations are in the nM to uM range. In addition, mouse serum or IgG can be added either as a pretreatment or as a component of the reaction to bind the potential inhibitor.

### Types of Epitopes

Conformational epitopes play a role in many biological processes, including, but not limited to, the complement cascade, antibody binding, receptor binding, enzyme regulation and signal transduction. Conformational changes that produce conformational epitopes are likely to occur whenever high affinity interactions take place between macromolecules. Some of these conformational epitopes may appear in the interior regions of the macromolecules, while others may appear on or near the surface of the macromolecules. Those conformational changes that can be recognized by a secondary binder are the subject of embodiments of the present technology. The binders of conformational epitopes can be isolated from protein display or aptamer libraries using standard panning and enrichment procedures.

The conformational epitopes used to generate a signal or initiate and propagate a chain reaction can occur naturally in the structure of the binder; or can be modifications to the structure of a binder, such as being strategically inserted into the structure of the binder; or conjugated to the structure of the binder; or an epitope in the structure of the binder can be hidden by an associated molecule that is modulated by interaction of the binder with the analyte under assay. Note that each type of binder, for example, antibody, antibody fragment, aptamer, et al., can have a naturally occurring conformational epitope or be modified with an insert or a conjugate or be modulated with an associated molecule.

Naturally occurring conformational epitopes are those that are an intrinsic component of a binding molecule. The epitope is hidden or the exact epitope shape does not exist until a binder interacts with its specific binding partner and, in the process, movements within the interacting molecules create the epitope or expose the epitope. Naturally occurring epitopes are identified when the complex of an analyte under assay and its specific binding partner are used to isolate secondary binders that are specific for epitopes formed when the analyte and binder interact. Secondary binders can be isolated from a protein display library or aptamer library. In addition, antigen antibody complexes can be used as an immunogen to produce an immune response with the subsequent formation of a hybridoma that produces a secondary binder.

There are several known and well studied naturally occurring conformational epitopes in the framework of antibodies that may be used in the practice of the present technology. For example, the C1Q binding site and the FcgR1 binding site of an antibody molecule. The C1Q binding site is essential to initiate the complement cascade. The FcgR1 binding site is recognized with high affinity by certain cells that modulate immune response. These sites are physically located close to each other and reside in the Fc region on an antibody. The C1Q binding site is in the CH2 domain and the FcgR1 binding site is at the junction of the CH2 and CH3 domain. It is estimated that the distance between the sites is ˜1-2 nm. The C1Q binding site and FcgR1 binding site may be used as epitopes for binding of a secondary binder of the present technology.

Inserted epitopes can be modifications made to the nucleic acid sequence of a primary binder, secondary binder, linker or an amplifier, that change the amino acid sequence of the primary binder, secondary binder, linker or amplifier in an attempt to create a conformational epitope that can be used to generate a signal or amplify a signal used to detect a biomarker in a sample. Examples of an inserted epitope include the strategic placement of a random peptide sequence or a known immunogenic sequence, for example, a FLAG tag, c-Myc tag or a His-tag. Inserted epitopes may also be short nucleic acid sequences inserted into larger nucleic acid sequences in an attempt to create a conformational epitope. The insertion is carried out by standard molecular biology techniques using either DNA or messenger RNA as the starting material. An insertion can be made into any of the binders listed above obtained from any of the sources listed above.

Conjugated epitopes can be molecules covalently coupled to a binder. The epitope can be for example, a peptide, carbohydrate, lipid, nucleic acid, hapten, polymer or any molecule conjugated to a primary binder, secondary binder, linker or amplifier; the function of which is to participate in a conformational change that modulates a conformational epitope that can be used to generate or amplify a signal used to detect a biomarker in a sample. Examples of a conjugated epitope include, for example, a FLAG-tag, c-Myc-tag, His-tag and biotin. Conjugated epitopes are covalently linked to the binder. To enable the strategic placement of the conjugate, it may be desirable to insert a track of amino acids as a preferred binding site for conjugation. For example, a cysteine track can be inserted at a strategic site in a binder to enable a cross linking reagent like SPDP that reacts with a sulfur atom. Conjugates can be made with any of the binders listed above, obtained from any of the sources listed above.

It is also possible to create a conformational epitope through the interaction of an associated molecule that hides an epitope until modulated by a binding event. Associated molecules are used to cover naturally occurring, conjugated or inserted epitopes. The associated molecules are held in place by electrostatic, hydrophobic, hydrogen bonding and Van der Waals forces. The interaction is modulated either by the primary binder interacting with the analyte under assay, a secondary binder or linker interacting with the primary binder or the amplifier interacting with the primary binder. Examples of an associated molecule include, for example, binders selected from an antibody or peptide library that are associated with a primary binder in the absence of the analyte under assay but are dissociated upon interaction with the analyte under assay or molecules that are computationally designed to interact with a site on a binder but dissociate when the binder interacts with its specific target molecule. An associated molecule can be designed for any binder including those listed above which are obtained from any of the sources listed above.

### Isolation and Characterization of Primary Binders and Secondary Binders

The following sections describe methods for selecting primary binders, secondary binders, linkers and amplifiers with naturally occurring conformational epitopes. The binders may come from many sources as described above in the section “Types of Binders”. The example below focuses on an antibody fragment display library expressed on phage. The steps outlined below, however, can also be used to screen other libraries that express potential reagents for use in embodiments of the present technology.

### Isolation of Primary Binders

A primary binder is selected to have affinity and specificity for the analyte under assay (AUA) and produce a conformational epitope or epitopes upon binding the analyte under assay. It is a requirement that a primary binder has at least one conformational epitope to be used in the FRET or the amplification format. Conformational epitopes are hidden within the superstructure of the primary binder but become exposed upon binding the specific target. The primary binder may be, for example, a Mab or a polyclonal antisera or an antibody fragment isolated from a library, see section above entitled “Types of Binders” for a more thorough discussion of alternatives. The specific example below envisions a phage display library expressing a Fab fragment on its surface.

### Reactivity for Antigen

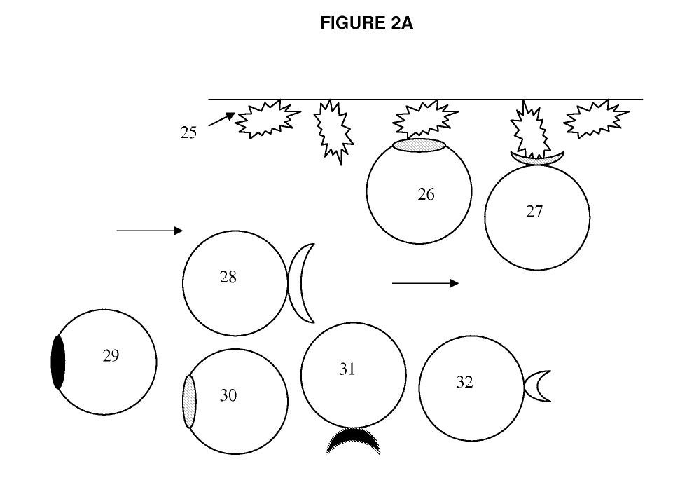

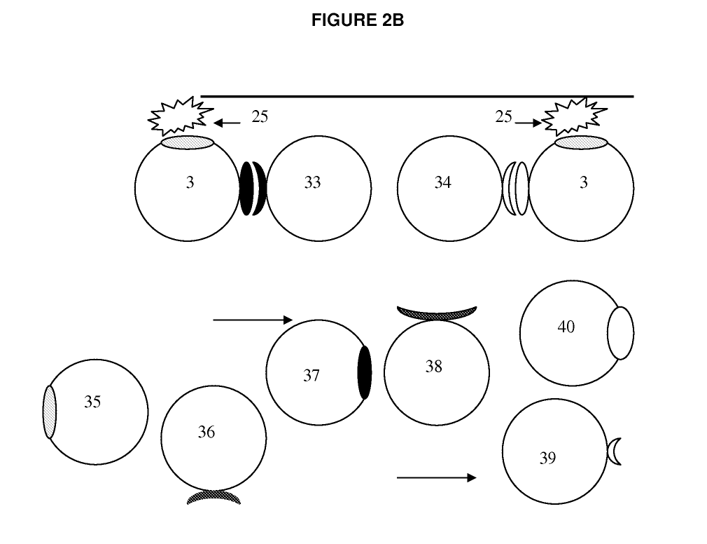

To isolate a primary binder the analyte under assay is immobilized to a solid phase. The immobilization can be by, for example, passive adsorption, covalent linkage or mediated by a binding partner. For example, the analyte under assay might have a tag incorporated at its carboxyl terminus that could be used to bind to an anti-tag antibody immobilized to the solid phase. The solid phase coated with analyte under assay and the library members are brought together and incubated for an appropriate period of time. The solid phase is washed to remove unbound or weakly associated library members. The bound library members are dissociated from the solid phase, collected and amplified by replication in an appropriate host cell. The progeny of the amplification are collected and cycled through additional rounds of binding, washing, dissociation and amplification. The process is repeated as required. See, for example, Eisenhardt et al. incorporated by reference in its entirety. FIG. 2A diagrams the general process to select primary binders from an antibody display library. The analyte under assay 10 is immobilized on a solid phase support 25. Members of the library 26-32 are passed over the immobilized analyte. Those with sufficient affinity for the analyte 26, 27 bind during the process and remain bound during steps designed to remove weakly bound or non-specifically bound members.

### PDL Selection Process

Isolation of primary binders may take place by employing either positive and/or negative selection schemes, for example, as disclosed in Sidhu 2000 and Rodi et al,. 1999, incorporated by reference in their entirety.

In some embodiments, positive selection is based on isolation of phage display clones that have high affinity for a specific antigen. The selection process requires that the phage display clones be bound to the antigen with high affinity. Unbound clones or weakly associated clones must be removed by washing. The selection process can take place on magnetic particles to facilitate separation. In the case of antigens displayed on cells, the cell can be tethered to magnetic particles to isolate clones. Recombinant antigen can be used as an antagonist or competitor to enrich for specific binders.

In some embodiments, a depletion schemes may be used to select phage display clones. Because phage libraries are composed of millions and potentially billions of members and have binders to almost every molecule shape, it may be advantageous to deplete the library of binders to specimen matrix components, cells and any solid surfaces used to carry out the isolation at the beginning of the procedure. An exemplary screening strategies includes 3 rounds of positive selection with bound phage eluted with recombinant antigen as competitor followed by elution with low pH. Next, the selected phage clones undergo depletion with solid phases, final matrix components and cells followed by positive selection with bound phage eluted with recombinant antigen as competitor followed by elution with low pH. Each round is defined by positive or negative selection of the library followed by washing, eluting and amplifying in E. coli.

The specificity of the primary binders isolated above can be determined by performing the reaction of primary binder and analyte under assay in the matrix in which the analyte under assay will appear in the final assay. For example, if the analyte under assay will be detected in a serum sample, the binding reaction should be performed in the presence of serum to determine if there is any interference with the binding event. In addition, the primary binders should be tested for reactivity to molecules or proteins that have a similar molecular structure to the analyte under assay. No interference with the binding reaction or reaction with any matrix component or closely related protein or molecular structure should be observed.

The binding position and relative affinity of the primary binders can be assessed by competitive reactions. To assess the relative position of primary binders on the analyte under assay, each primary binder isolate needs to be labeled with a signal generating molecule either directly or indirectly through a binding partner that carries the signal generating molecule. Examples of a signal generating molecule include, but are not limited to, a fluorescent or luminescent label, or an enzyme tag that catalyzes the formation of a signal generating molecule. Each labeled primary binder is then incubated with the analyte under assay in the presence of increasing amounts of an unlabeled primary binder. If an unlabeled isolate interferes with the binding of the labeled isolate, it is an indication that the two isolates bind the analyte under assay at the same site or at sites that are proximal on the analyte under assay. Alternatively, two labeled primary isolates can be simultaneously incubated with the analyte under assay. If the signals are additive, that is an indication that the isolates react at different sites on the analyte under assay. To assess the relative affinity of isolates for the antigen, the concentration of each isolate stock solution is determined. For example, this can be done by titration using an ampicillin sensitive host and a B-lactamase expressing phagemid. Quantification of the stock solution can also be done by plaque assay. A constant amount of labeled analyte under assay is added to dilutions of each isolate to be tested. After an appropriate period of incubation, the labeled analyte under assay-primary binder complexes are captured on a solid phase coated with anti-primary binder. The lowest dilution of an isolate that captures 50% of the label establishes relative affinity. Alternatively, free and bound label could be measured after chromatographic separation.

### Identification of Naturally Occurring Conformational Epitopes in Primary Binders

Any binder that binds to an analyte under assay-primary binder (referred to hereafter as AUA-PB) complex but does not bind the analyte under assay alone or the primary binder alone is a secondary binder. A secondary binder that is reacting with a conformational epitope on the primary binder defines a conformational epitope on the primary binder. A primary binder that has at least one secondary binder may be appropriate to initiate the amplification reaction, bind a linker for the FRET format or amplification format or be a candidate for the FRET format or the linear amplification FRET format. In some embodiments, secondary binders that are binding to CEs on the antigen or at the antigen-primary binder interface are removed from the pool of binder candidates.

It is an advantage to use a Mab or a complete antibody to form the AUA-PB complex, as the bait, during the initial isolation of secondary binders from a library. The presence of the CH2 and CH3 domain increases the probability of finding secondary binders. In fact, there are several known and well characterized CEs in the Fc regions of antibodies, for example, the C1Q and the FcgR1 binding sites.

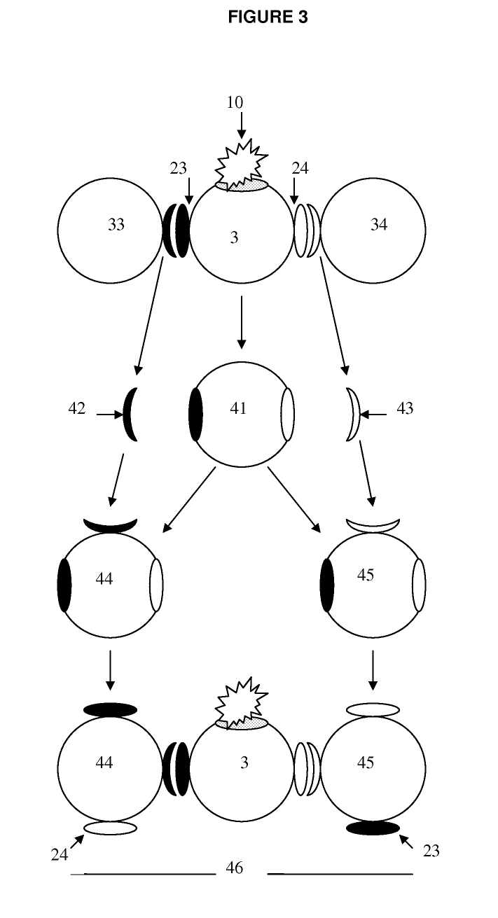

The isolation of two secondary binders to a primary binder that do not interfere with each other's binding defines the presence of two non overlapping conformational epitopes on a primary binder, which defines a superstructure that may be appropriate to serve as a labeled amplifier molecule, a FRET primary binder, a FRET linker or a FRET linear amplification molecule. Not to be bound by any particular theory, it is anticipated that the superstructure of a primary binder with more than one non-overlapping conformational epitope can be used to insert variable regions or CDRs or binding domains from other binders (FIG. 3). It is also anticipated that the epitopes may be <10 nm apart or reside within <10 nm of a stable epitope and may be used in FRET format development.

FIG. 3 diagrams the general process to create an amplifier binder using the superstructure of a primary binder with two known non-overlapping conformational epitopes. The binding domains from two different secondary binders 42, 43 with affinity and specificity for the epitopes on the primary binder are inserted into the superstructure of the primary binder. 41 is the superstructure of primary binder minus binding site, 44 and 45 are engineered amplifier binders, and 46 is a primary binder with exposed epitopes bound to engineered amplifier binders with exposed epitopes. This process is analogous to antibody humanization used in biopharmaceutical development.

### Assessment of Primary Binders for Conformational Epitopes

### Secondary Binder—Isolation and Assessment—Binding Position, Relative Affinity and Specificity

Isolation—To determine if a primary binder displays a naturally occurring conformational epitope, a primary binder is incubated with its specific analyte under assay and the resulting AUA-PB complex is immobilized on a solid phase support or captured onto the solid phase by a binder to a second site on the analyte under assay or formatted as a solution assay. See, for example, Eisenhardt, incorporated by reference in its entirety. The solid phase coated with AUA-PB complex is then exposed to a library of binders, see, for example, FIG. 2b.

See FIG. 2B, which diagrams the general process to select secondary binders from an antibody display library. The analyte under assay 25-primary binder 3, AUA-PB, complex is immobilized on a solid phase support. Members of the library 35-40 are passed over the immobilized complex. Those with sufficient affinity for conformational epitopes on the AUA-PB complex (33 and 34) bind during the process and remain bound during steps designed to remove weakly bound or non-specifically bound members.

Library members that bind to stable epitopes, non-conformational epitopes, on the primary binder or analyte under assay can be removed before passage over the AUA-PB complex. One method to remove these library members includes passing the library over a solid phase with immobilized primary binder or analyte under assay before the library is passed over the AUA-PB complex. Binders that bind to the AUA-PB complex but not to the primary binder alone, analyte under assay alone or possibly a conformational epitope on the analyte under assay are secondary binders that can be assessed for utility in the_FRET format (FIG. 1A) or engineered into amplifiers for the amplification format (See FIGS. 1B, 1C). Secondary binders are enriched by repetitive cycles of binding, washing, releasing and enriching. Cycles of binding, washing, releasing and enriching are repeated as needed. (See section above entitled PDL Selection Process.)

### Assessments

Secondary binders for the FRET format are assessed for binding position, affinity for the primary binder, specificity, ability to be labeled with a donor or acceptor fluorophore and ability to generate a FRET signal. Secondary binders that are being assessed as potential candidates for the amplification format are screened for binding position on the primary binder, specificity, affinity for the primary binder, ability to be labeled with a signal generating molecule and ability to sustain the amplification reaction.

To assess the performance of binders the Fab expressed on the phage isolate needs to be expressed as a protein in solution. The process for expression is well known to one in the art. (See, for example, http://www.creative-biolabs.com)

### Binding Position—Assuring the Binding Position is on the PB

Evidence that the secondary binder is binding to the primary binder can be obtained by reacting the AUA-PB complex with binders to stable epitopes on the primary binder followed by reaction with labeled secondary binder. Interference with the binding of the labeled secondary binder is an indication that the conformational epitope is on the primary binder. Evidence that the secondary binder is binding to the analyte under assay can be obtained by reacting the AUA-PB complex with other primary binders that bind at different sites on the analyte under assay followed by reactions with labeled secondary binder. Interference with the binding of the labeled secondary binder is an indication that the conformational epitope is on the analyte under assay. Alternatively, evidence for the location of the secondary binder's binding site can be obtained by oxidation or chemical modification of the AUA-PB complex followed by dissociation of the complex, separation of modified analyte under assay from modified primary binder, followed by reformation of two AUA-PB complexes; one complex consisting of modified analyte under assay and unmodified primary binder and the other unmodified analyte under assay and modifier primary binder. Each AUA-PB complex is then reacted with labeled secondary binder. Interference with binding because of modification is an indication that the secondary binder is binding to the modified portion of the complex.

### Relative Binding Position on the PB

In one embodiment, assessment of whether secondary binders are reacting at the same or at different sites on the primary binder can be determined by labeling each secondary binder with a signal generating molecule. An appropriate amount of a labeled secondary binder is incubated with the AUA-PB complex and increasing amounts of an unlabeled secondary binder. If there is no decrease or interference in the signal level with increasing amounts of an unlabeled secondary binder, this indicates that the secondary binders are reacting at different sites on the primary binder. Alternatively, combinations of labeled secondary binders can be incubated with AUA-PB complex to determine whether the signals are additive. If they are additive, this is an indication that the secondary binders are reacting at different sites on the primary binder.

### Assessing Secondary Binders for Relative Affinity

### Relative Affinity

To assess the relative affinity of secondary binders for an AUA-PB complex, each secondary binder can be tagged with biotin for example, and the concentration of each tagged secondary binder stock solution determined by plaque assay. A constant amount of analyte under assay and primary binder (one or the other needs to be labeled with a signal generating molecule) is added to reaction tubes and incubated for an appropriate period of time to form a signal labeled -AUA-PB complex. Next, dilutions of each tagged secondary binder are made and added to the reaction tubes. After further incubation the labeled analyte under assay-primary binder-tagged secondary binder complexes are captured onto an anti tag coated solid phase, for example, avidin. The lowest dilution of tagged secondary binder that captures 50% of the label complex establishes relative affinity. (Alternatively, free and bound label can be measured after chromatographic separation.)

### Assessment of Secondary Binder Specificity

### Specificity

The specificity of the secondary binders is assessed by reaction with analyte under assay alone, primary binder alone, final matrix components and other primary and secondary binder isolates. No interference or reaction should be observed with any of these reagents.

### Assessing Secondary Binders—Fitness for Purpose—for the FRET Format, Amplification Format or Multi-Assay Utility

Screening a library for secondary binders will lead to a collection of molecules that can be used as components in the amplification or FRET format or act as building blocks or precursors that are engineered into components for the amplification or FRET format. In addition to secondary binders to conformational epitopes, the screening process will also isolate binders to stable epitopes on the primary binder. The following section summaries the paths that the secondary binders can take as they move forward in some embodiments of the present technology.

### Paths Forward

When a Fab secondary binder is isolated from a PDL and the bait used to isolate the Fab was an AUA-PB complex in which the PB was a Mab or whole antibody, the Fab secondary binders may be binding in the Fab portion of the primary binder or in the Fc portion of the primary binder. Re-screening with an AUA-PB complex in which the primary binder is a Fab will help identify the exact position.

In some embodiments, 2 Fab secondary binders properly spaced can form a FRET pair. The pair can bind in the Fab or Fc region of the primary binder.

In some embodiments, 2 Fab secondary binders that bind a Fab primary binder can be engineered into amplifiers by the method described in FIG. 3.

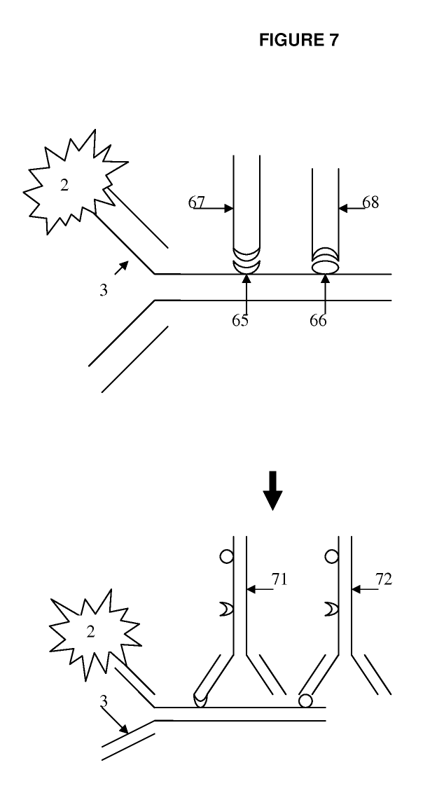

In some embodiments, 2 Fab secondary binders that bind a whole antibody primary binder in the Fc region can be engineered into amplifiers by inserting a Fc region into their structure by the method described in FIG. 7.

When assessing a collection of secondary binders the following guidelines need to be observed:

FRET Format—To be a primary binder for the FRET format, it is necessary to have two non-overlapping epitopes that reside <10 nm apart. At least one of the epitopes must be a conformational epitope. The two epitopes are docking sites for a FRET pair. Secondary binders that are candidates to become a member of a FRET pair must be specific for the primary binder, not interfere with each other's binding, bind to sites <10 nm apart on the primary binder, carry a donor or acceptor molecules, generate a FRET signal and bind with appropriate affinity. Finding a FRET pair is accomplished by testing all possible combinations of secondary binders for FRET signal generation. The FRET pair can have one member react with a stable epitope on the primary binder. Binders to stable epitopes can be isolated by using a primary binder as bait in the absence of specific antigen.

Amplification Format—In some embodiments, the secondary binder has at least two conformational epitopes to be an amplifier binder. If some of the amplifier binders contain only one functional conformational epitope the reaction will still proceed. An efficient way to engineer amplifier binders from secondary binders is to start with a primary binder that has two non-overlapping secondary binders. An embodiment of the present technology includes a primary binder that has affinity and specificity for its target analyte and has more than one non-overlapping secondary binder. The superstructure of this primary binder is a good candidate to use to construct amplifiers that possess more than one conformational epitope. The primary binder's superstructure can be used to insert the binding domain, variable regions or the CDRs from secondary binders (FIG. 3). The product of the insertion will have the binding specificity of the secondary binder in a superstructure known to produce more then one conformational epitope. If the two resulting hybrid amplifier molecules retain affinity and specificity for their respective conformational epitope on the primary binder, and are shown to be self reactive and cross reactive when presented with an analyte under assay-primary binder-secondary binder (hereafter referred to AUA-PB-SB), complex, this constitutes a functional analysis system of the present technology. There will be many primary binders to an analyte under assay and each of those primary binders will have secondary binders; this process can be repeated until the desired result is obtained. Secondary binders that are engineered into amplifier binders are assessed for specificity, non interference with each other, affinity and ability to be labeled.

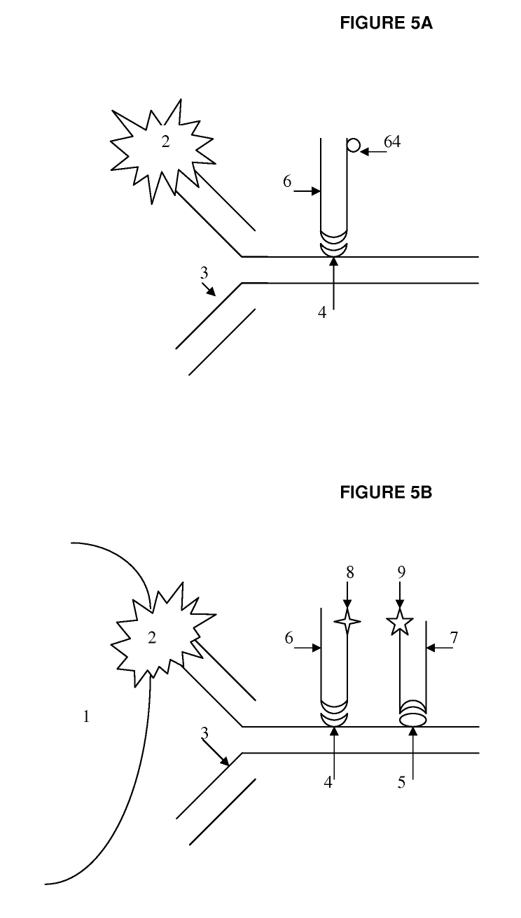

Multi Assay Utility—Whenever a secondary binder binds to the CL, CH1 or Fc region of the primary binder, that secondary binder has the potential to bind other primary binders that share the same CL, CH1 or Fc regions. The CL, CH1 and Fc regions of an immunoglobulin subclass, for example, IgG-1, are common among members of that subclass; therefore, secondary binders that bind one member of the subclass may bind other members of the subclass. Any existing monoclonal or polyclonal antibody, therefore, can potentially be used in the amplification (FIG. 5A) or FRET format (FIG. 5B) using a secondary binder or binders with specificity for a conformational epitope presented in the antibody's subclass. This is an obvious advantage for accelerated product development. The C1Q binding site and the FcgR1 binding site are well known conformational epitopes in the Fc portion of a mouse or human antibody. Secondary binders, specific for the C1Q binding site or the FcgR1 binding site can be used to couple existing mouse or human antibodies into the FRET or amplification format. It is possible that C1Q and FcgR1 binding site binders selected from a human Fab PDL may show cross reactivity with circulating human immune complexes. It may therefore be an advantage to use a mouse Fab PDL to select the C1Q and the FcgR1 binding site binders.

FIG. 5A diagrams a general method to utilize an existing binder: a mouse monoclonal antibody, a polyclonal antibody or an aptamer to initiate the amplification reaction. In the example presented, a mouse IgG monoclonal antibody (primary binder 3) binds its specific antigen 2 which exposes a conformational epitope 4 in the CH2-CH3 constant region of the antibody. A secondary binder 6, specific for the conformational epitope on the primary binder, binds the exposed epitope which produces a conformational epitope (on the secondary binder) 64 in its superstructure. The exposed epitope is recognized by an amplifier binder molecule which initiates the amplification reaction, (not shown). The secondary binder in this case is acting as a linker molecule between the primary binder and the amplifier. Alternatively an amplifier molecule with specificity for the exposed epitope can directly initiate the amplification reaction. Any primary binder presenting an epitope recognized by an amplifier binder or a secondary binder that exposes an epitope recognized by an amplifier binder can be used to initiate the amplification reaction.

FIG. 5B diagrams a general method to utilize any primary binder 3 that exposes two epitopes 4, 5 that are less than 10 nm apart, wherein at least one must be a conformational epitope, to initiate the FRET reaction. In the example presented, a mouse IgG monoclonal antibody 3 binds to its specific antigen 2 on the analyte under assay 1 and exposes two conformational epitopes 4, 5 in its Fc region. Secondary binders 6, 7 with specificity for the conformational epitopes are labeled with a FRET pair, donor molecule, 8, and acceptor molecule, 9. Excitation of the complex with an appropriate wavelength produces a FRET signal.

### Assessing Primary Binders

FRET format—The primary binder in the FRET format must have affinity and specificity for the target analyte and have two or more non-overlapping secondary binders, at least one of the secondary binders must react with a conformational epitope. The non-overlapping secondary binders are labeled with a donor or an acceptor fluorophore-pair and various combinations tested to see if they produce a FRET signal. Optimization of fluorophore concentration and labeling chemistry are necessary.

Amplification format—The primary binder for the amplification format must have affinity and specificity for the target analyte and at least one conformational epitope that is recognized by an amplifier molecule or a linker molecule.

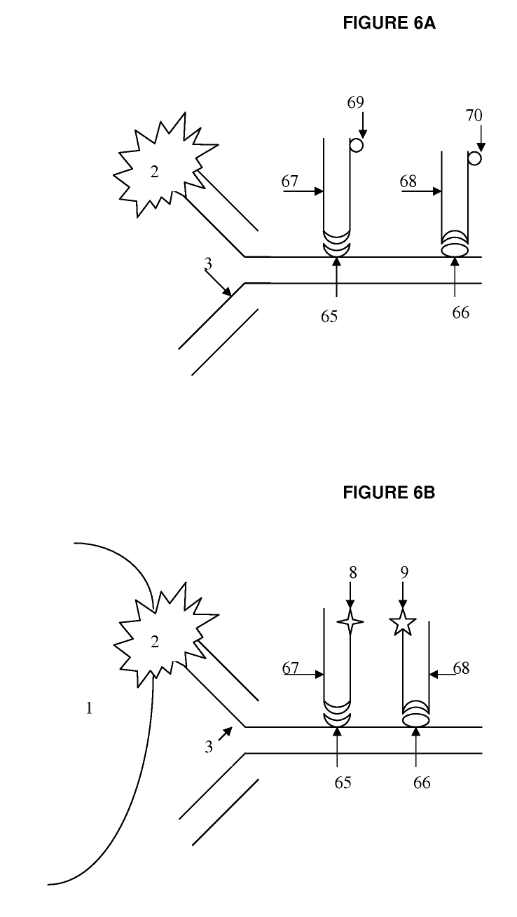

Any mouse or human IgG molecule that presents the C1Q binding site upon binding its specific antigen can be coupled into the amplification format by way of the C1Q binding site 65 (See FIG. 6A). For example Ag-Mab complex—C1Q binding site exposed—amplifier molecule or linker molecule with mouse anti-C1Q binding site specificity. 67 is a secondary binder with anti-C1Q binding site specificity, 68 is a secondary binder with anti-FcgR1 binding site specificity, while 69 and 70 are conformational epitopes on 67 and 68, respectively.

Any mouse or human antibody that presents the FcgR1 binding site upon binding its specific antigen can be coupled into the amplification format by way of the FcgR1 binding site 66 (FIG. 6A). For example, (Ag-Mab complex—FcgR1 binding site exposed—amplifier molecule or linker molecule with mouse anti-FcgR1 binding site specificity).

The selection of two Fab secondary binders, one with anti-C1Q binding site specificity and the other with anti-FcgR1 binding site specificity, may be used for the FRET format of the present technology since those binding sites are <2 nm apart (FIG. 6B).

The selection of two Fab secondary binders, one with anti-C1Q binding site and the other with anti-FcgR1 binding site specificity, can be made into amplifiers by inserting the CH2-CH3 domains onto the carboxyl terminus of the CH1 domain (FIG. 7). 71 and 72 are antibodies with specificity for the C1Q and FcgR1 binding sites engineered by inserting a CH2-CH3 domain on the carboxyl terminus of two Fabs with specificity for the C1Q and FcgR1 binding sites

The selection of two Fab secondary binders to a Fab primary binder can be made into amplifiers by the method described in FIG. 3.

The selection of two Fab secondary binders that bind CEs in the Fc portion of a whole antibody primary binder can be made into amplifiers by inserting the CH2-CH3 domains of that primary binder onto the CH1 domains of the two Fab secondary binders. (the process used in FIG. 7).

The selection of any two non-overlapping secondary binders to CEs that are within 10 nm of each other fills the requirements of the FRET format.

The selection of any two non-overlapping secondary binders that are within 10 nm of each other, at least one secondary binder recognizes a CE, fills the requirements of the FRET format.

FIG. 6A diagrams an exemplary general method to utilize the C1Q binding site 65 or the FcgR1 binding site 66 displayed in the Fc region of certain human and mouse antibodies 3 to initiate the amplification reaction. In the example presented, a mouse IgG monoclonal antibody 3 binds to its specific antigen 2 and exposes two conformational epitopes the C1Q binding site 65 and the FcgR1 binding site 66 in the Fc region of the antibody. Fab secondary binders 67, 68 to the conformational epitopes are isolated from a PDL. One secondary binder has specificity for the C1Q binding site 67 and one has specificity for the FcgR1 binding site 68. In addition, the secondary binders have a conformational epitope 69, 70 in their superstructure that is recognized by an amplifier molecule, not shown, to initiate the amplification reaction. In this case, the secondary binders are acting as linkers between the primary binder and the amplifier. Any primary binder displaying the C1Q or the FcgR1 binding site can be coupled into the amplification reaction by way of the linkers. Alternatively, the Fab secondary binders in this example can be engineered into amplifier binders by the process described in FIG. 3 or into whole antibody amplifiers by the process described in FIG. 7. The amplifier binders produced by the engineering process will directly bind the primary binder and initiate the amplification reaction; no linker molecule is necessary. It should be noted that the whole antibody amplifiers engineered above will expose the C1Q binding site and the FcgR1 binding site as their conformational epitopes, just like the parental primary binder.

FIG. 6B diagrams a general method to utilize two known conformational epitopes, the C1Q binding site 65 and the FcgR1 binding sites 66 that reside ˜2 nm apart to generate a FRET signal. In the example presented, a mouse IgG monoclonal antibody 3 (primary binder) binds to its specific antigen 2 presented on the surface of a cell 1 (the entity under assay) and in so doing exposes two conformational epitopes, the C1Q binding site In some embodiments, 65 and the FcgR1 binding site 66 in the Fc portion of the antibody. Fab secondary binders that are specific for the C1Q binding site 67 and the FcgR1 binding site 68 are isolated from a PDL. One secondary binder is labeled with a donor molecule 8 and the second secondary binder is labeled with an acceptor molecule 9 from a FRET pair. When the labeled secondary binders 67, 68 are bound to the primary binder epitopes 65, 66, the pair is properly oriented to generate a FRET signal. The solution containing the epitope-primary binder-secondary binders complex is illuminated with a wavelength that excites the donor molecule. The excitation energy is transferred to the acceptor molecule which emits a photon at a specific wavelength that is detected by an appropriate optical system. The reagents developed in this process can be used with any primary binder (any mouse antibody) that displays the C1Q and FcgR1 binding sites upon binding its specific ligand. The process also works with a secondary binder to a stable epitope and either the C1Q binding site secondary binder or the FcgR1 binding site secondary binder as long as they reside <10 nm apart and will produce a FRET signal.

One embodiment of the present technology is described in FIG. 7. The amplifier binders 71, 72 produced by the engineering process directly bind the primary binder 3 and initiate the amplification reaction; no linker molecule is necessary. FIG. 7 diagrams a method to engineer whole antibody amplifier molecules from Fabs with anti-C1Q binding site 65 specificity and anti-FcgR1 binding site 66 specificity. In this example, the primary binder 3 is a mouse IgG monoclonal antibody that is bound to its specific antigen 2 and in so doing exposes two conformational epitopes, the C1Q binding site 65 and the FcgR1 binding site 66 in the Fc portion of the antibody. Fab secondary binders to the C1Q binding site 67 and FcgR1 binding site 68 are isolated from a mouse PDL. Once the Fabs are isolated, the Fc region of the primary binder can be inserted on the CH1 carboxyl terminus of the Fabs. In so doing, two amplifier molecules are created that will also expose the C1Q and FcgR1 binding sites upon binding their respective targets. The process will work for any two non-overlapping conformational epitopes identified in the Fc region of a primary binder. In this example, it is assumed that the Fabs and the inserted Fc are from the same species. However engineering a chimeric molecule is also possible.

The processes described above generate reagents that fill all the requirements of the FRET format and the amplification format. The processes can be generalized to use any conformational epitope in the Fc of an immunoglobulin. Detailed selection process described below.

### Isolating Secondary Binders to Mouse IgG C1Q and FcgR1 Binding Sites

Mouse IgG molecules are reacted with their specific binding partner. The antigen may be free in solution or displayed on the surface of a cell. See, for example, Sidhu 2000; Rodi et al., 1999, incorporated by reference in their entirety.

Isolation of secondary binders may take place by employing positive and/or negative selection schemes. Positive selection is based on isolation of phage display clones that have high affinity for the conformational epitopes on IgG mouse monoclonal antibody that is bound in complex with its specific antigen. The selection process requires that the mouse antibody be bound to the antigen in order to expose the conformational epitopes. In addition, unbound clones or weakly associated clones must be removed by washing. The complexes can be immobilized on magnetic particles to facilitate separation. In the case of antigens displayed on cells, the cell can be tethered to magnetic particles and the antigen-antibody complexes displayed on the cell surface. In the case were the CE is known, the natural binder can be used as an antagonist or competitor to enrich for specific binders. Depletion schemes—Because phage libraries are composed of millions and potentially billions of members and have binders to almost every molecule shape, it may be advantageous to deplete the library of binders to specimen matrix components, immunoglobulins that are not in complex with antigen and solid phases used to carry out the isolation at the beginning of the procedure.

### Screening in 4 Arms—See, Rodi et al., 1999

The following describes a screening method to isolate phage specific for binding to the C1Q binding site as disclosed in Rodi et al. 1999, incorporated by reference in its entirety.

3 rounds of positive selection with bound phage eluted with recombinant C1Q as competitor followed by elution with low pH.

3 rounds of positive selection with bound phage eluted with recombinant CD64 as competitor followed by elution with low pH.

Depletion of phage clones with uncoated magnetic particles, human whole blood components and unbound mouse IgG followed by positive selection with bound phage eluted with recombinant C1Q as competitor followed by elution with low pH.

Depletion of phage clones with uncoated magnetic particles, human whole blood components and unbound mouse IgG which is followed by positive selection with bound phage eluted with recombinant CD64 as competitor followed by elution with low pH.

Each round is defined by positive or negative selection of the library followed by washing, eluting and amplifying in E. coli.

Using the procedure outlined above, binders to the C1Q and FcgR1 sites on a mouse IgG molecule can be isolated and characterized for affinity and specificity. Once these binders are found to have appropriate performance characteristics they can be consider multi assay reagents. They can be used in any conformational epitope format with a mouse immunoglobulin that displays the appropriate epitope upon binding its specific antigen. The binders can be labeled with a donor acceptor pair to produce a FRET signal on, for example, any mouse IgG antibody that displays the epitopes, (FIG. 6b). In addition, these Fabs can be engineered into whole antibodies by inserting the CH2-CH3 domains at the carboxyl terminus of the CH1, domain (FIG. 7). Any two CEs in the mouse antibody superstructure, for example, that are not cross reactive with human antigen-antibody complexes and meet CE format requirements can be used as a multi assay reagent for integration of existing mouse antibodies. It is possible that some IgG subclasses may need their own specific reagents.

Construction of a Primary Binder or an Amplifier with an Inserted Epitope

A further embodiment of the present technology includes strategically inserting the nucleic acid sequence coding for a peptide of known reactivity or of unknown reactivity into the nucleic acid sequence of the phage isolate expressing a primary or secondary binder. The inserted epitope will be expressed in the progeny of the recipient primary or secondary binder. Examples of an inserted peptide of known reactivity include, but are not limited to, a FLAG-tag, c-Myc-tag or any peptide that has a known binding partner. An example of an inserted peptide of random sequence is a highly charged peptide that is likely to attract a binding partner. If the primary binder and amplifiers are structurally nucleic acids, the strategic or random insertion of nucleic acid sequence to create conformational epitopes is also possible.

Structural data show that several CDRs in an antibody, Fab, scFv or diabody undergo movement upon interaction with its specific ligand. The H3 loop has been particularly well documented (See, for example, Wilson and Webster). The carboxyl end of the H3 loop is tethered to the CH1 domain in a Fab by a stretch of amino acids. Similarly, the VH and VL domains of a scFv or a diabody are tethered by a short linker of amino acids. These transition or linker regions are potential sites to insert an epitope, since they may undergo movement with the H3 domain when the molecule binds its specific target.

The placement of an inserted epitope in a binder can be aided by precise structural data from X-ray crystallography and nuclear magnetic resonance. In addition, the design and positioning of an insert can be aided by computation methods (See, for example, Lippow, incorporated by reference in its entirety). Using information gathered from all available sources will help direct the placement of inserted epitopes.

Examples of appropriate sites for placement of an epitope in a Fab fragment can include, but are not limited to:

Extending from H3 or H2 or H1 or L3 or L2 or L1 or from a combination of CDRs

In the VH and VL or VH or VL

In the CH1 and CL or CH1 or CL

Between the VH and CH1 or VL and CL or VH and CH1 and VL and CL

In a peptide extending from the carboxyl end of the CH1

In a peptide extending from the carboxyl end of the CL

In a peptide extending from the carboxyl end of the CH1 and CL

Placement of epitope in a diabody can include, but are not limited to, for example:

Extending from H3 or H2 or H1 or L3 or L2 or L1 or from a combination of CDRs

In the VH and VL or VH or VL

In the linker between the VH and the VL

In the linker between the VL and the VH

Placement of epitope in a scFv can include, for example:

Extending from H3 or H2 or H1or L3 or L2 or L1 or from a combination of CDRs

In the VH and VL or VH or VL

In the linker between the VH and the VL

In the linker between the VL and the VH

Placement of an inserted epitope in a Mab or Ab can include, for example:

Extending from H3 or H2 or H1 or L3 or L2 or L1 or from a combination of CDRs

In the VH and VL or VH or VL

In the CH1 and CL or CH1 or CL

In the CH2 and CH3 or CH2 or CH3

Between the CH1 and CH2

Between the CH2 and CH3

Between the VH and CH1 or VL and CL or both VH and CH1 and VL and CL

In a peptide extending from the carboxyl end of the CH3

In a peptide extending from the carboxyl end of the CL

In a peptide extending from the carboxyl end of the CH3 and CL

### Insertion of a Protein-Protein Interaction

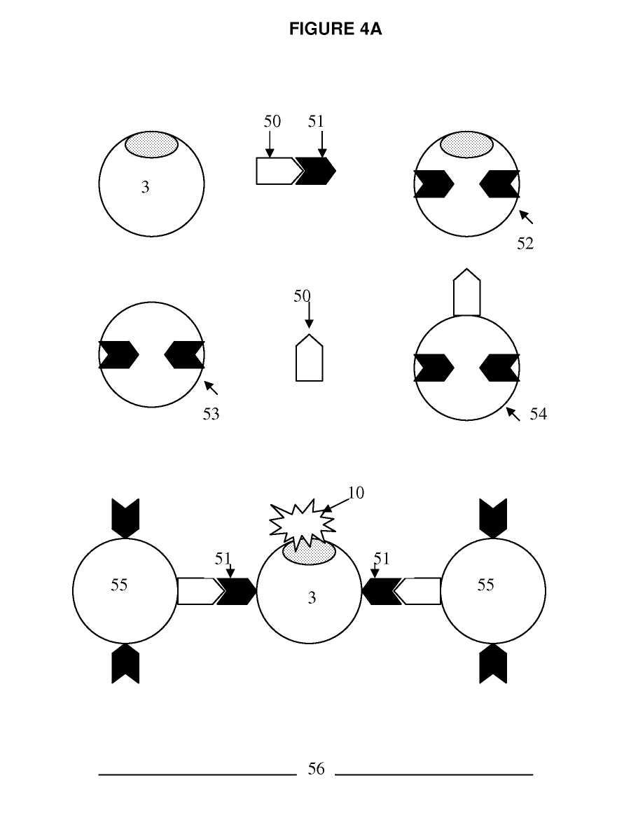

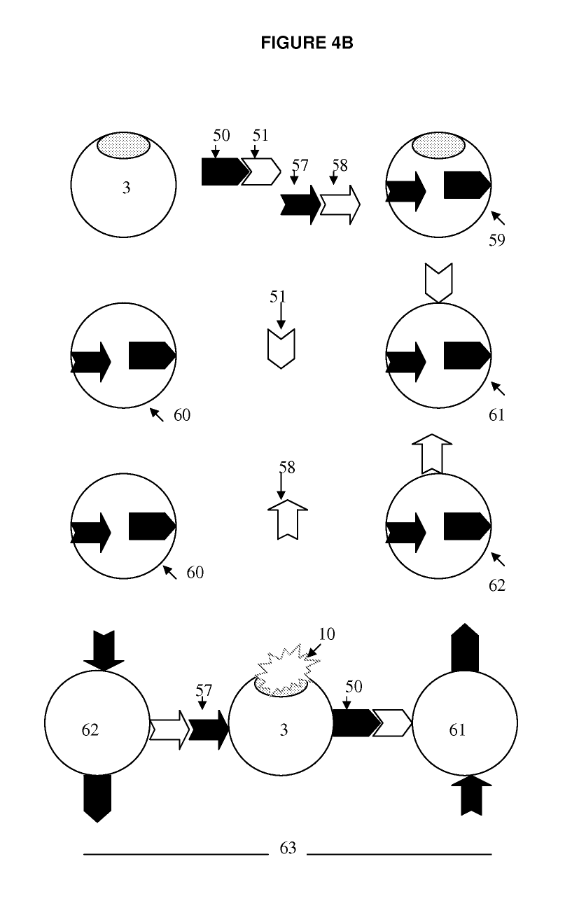

In another embodiment, primary binders and amplifiers can be created from known protein-peptide interactions, protein-protein interactions or, more specifically, protein domain-protein domain interactions. FIG. 4a diagrams the process of inserting one of the interacting domains at two conformational sites on a primary binder and then using that structure to engineer an amplifier. This process creates a primary binder and an amplifier with two identical epitopes. FIG. 4b diagrams the process of making a primary binder and two amplifiers using two different protein-protein interactions. One of the domains from each protein-protein interaction is inserted into a primary binder at two conformational sites. The second domain from each protein-protein interaction is inserted as the binding domain into two additional molecules that have the superstructure of the primary binder to form two amplifiers. This process creates a primary binder and two amplifiers with different epitopes.

FIG. 4A diagrams the general process to engineer an amplifier binder molecule from two domains known to participate in a protein-protein interaction, for example, a FLAG tag interacting with an anti-FLAG antibody. The primary binder with specificity for an analyte of interest has one domain from a known protein-protein interaction inserted at two conformational sites in the primary binder, for example, a FLAG tag inserted at the amino and carboxyl ends of the linker in a scFv. The binding site, CDRs, of the primary binder is then removed and the second domain from the known protein-protein interaction, for example, the CDRs of the anti-FLAG antibody inserted at the binding site. This creates an amplifier binder with specificity for the conformational epitopes inserted into the primary binder. 50 and 51 are domains known to participate in a protein-protein interaction, 52 primary binder with a protein domain inserted at two sites, 53 binding site removed from primary binder with a protein domain inserted at two sites/CDRs removed, 54 domain known to participate in a protein-protein interaction inserted at binding site, 55 engineered amplifier binders with exposed domains bound to primary binder with exposed domains, 56 primary binder bound to AUA with exposed domains and engineered amplifier binder with exposed domains bound to primary binder.

FIG. 4B diagrams the general process to engineer two amplifier binder molecules from the domains of two known protein-protein interactions. A primary binder with specificity for an analyte of interest has one domain from two known protein-protein interactions, inserted at two sites in the primary binder. The binding site, CDRs, of the primary binder is removed and the second domain from one of the known protein-protein interactions is inserted at the binding site creating one amplifier binder. The second domain from the second protein-protein interaction is inserted at the binding site creating the second amplifier binder. One can envision, for example, a FLAG-tag and a c-Myc-tag and their corresponding antibodies producing the reagents described above.

50, 51, 57, 58 are interacting domains from two different protein-protein interactions, 59 primary binder with a domain from two different protein-protein interactions inserted into superstructure, 60 binding site removed from primary binder with a domain from two different protein-protein interactions inserted into superstructure, 61 and 62 domains from two different protein-protein interactions inserted at the binding site of a primary binder with a domain from two different protein-protein interactions inserted into its superstructure, 63 primary binder bound to AUA with exposed domains and engineered amplifier binders with exposed domains bound to exposed domains of the primary binder.

Selection of a Primary Binder or an Amplifier with an Inserted Epitope

Primary binders with inserted epitopes can be isolated and selected as follows. Primary binders with inserted epitopes are incubated with an anti-epitope solid phase to remove isolates that display the epitope in the absence of the analyte under assay. For example, if the FLAG peptide is inserted into the primary binder an anti-FLAG antibody would be immobilized on the solid phase. Primary binders with inserted epitopes that are not captured by the solid phase are enriched by replication in an appropriate host. The enriched isolates are then incubated with the analyte under assay to form complexes which are then passed over a solid phase coated with anti-epitope. Isolates that are captured by the solid phase have an inserted epitope that is modulated by binding the analyte under assay and are isolated and enriched. Primary binders are assessed for specificity by reacting with matrix components, other secondary binders and closely related proteins. No reactivity should be seen with any of these reagents or molecules.

Secondary binders with inserted epitopes are isolated and selected as follows. Secondary binders with inserted epitopes are incubated with an anti-epitope solid phase to remove isolates that display the epitope in the absence of the AUA-PB complex. Secondary binders with inserted epitopes that are not captured by the solid phase are enriched by replication in an appropriate host. The enriched isolates are then incubated with AUA-PB complexes to form AUA-PB-SB complexes which are then passed over a solid phase coated with anti-epitope. Complexes that are captured by the solid phase have secondary binders with inserted epitopes that are modulated by binding the AUA-PB complex. The secondary binders are isolated and enriched. Secondary binders are assessed for specificity by reacting with matrix components, other secondary binders and closely related proteins. No reactivity should be seen with any of these reagents or molecules.

Construction of a Primary Binder and an Amplifier with a Conjugated Epitope

Another embodiment of the present technology includes producing a conformational epitope by chemically conjugating a peptide or reactive molecule to a binder. The conjugation site may be rationally or strategically designed or created by random reaction. Examples of coupling reagents that are routinely used to prepare such conjugates include, but are not limited to, heterobifunctional crosslinkers like SPDP (N-succinimidyl 3-(2-pyridyldithio)-propionate); m-maleimidobenzoyl N-hydroxysuccinimide ester; 1-ethyl-3-(3-diethylaminopropyl)carbodiimide (EDC); or succinimidyl 4-(N-maleimido-methyl)cyclohexane-1-carboxylate (SMCC). An example of strategic design is the insertion of a track of cysteine residues that will be used to tether a conjugate using SPDP. An example of rational design is the placement of a conjugate in a binder where the epitope and its site of interaction with the binder have been computationally determined by analysis of structure with the intent of modulating the epitope for purposes of amplifying a signal used to detect an analyte in a sample. If the primary binder and amplifiers are structurally nucleic acids, the strategic or random insertion of conjugates to create conformational epitopes is also possible.

Construction of a Primary Binder, Linker and an Amplifier Using an Associated Binder to Cover an Epitope that is Naturally Occurring, Inserted or Conjugated

Another embodiment of the present technology includes creating a conformational epitope by hiding or covering a naturally occurring, inserted or conjugated epitope on a primary binder, linker or amplifier with an associated molecule. The associated molecule binds to the epitope in the absence of its specific ligand but is dissociated when the primary binder or amplifier reacts with its specific ligand.

Associated molecules can be isolated by reacting a primary binder, linker or amplifier with a peptide, antibody or nucleic acid library, and enriching binders by repetitive cycles of binding, release and replication as described above. Alternatively, the primary binder, linker or amplifier can be used as an immunogen, and hybridomas developed that produce reactive antibodies that are used as is, or their binding domains inserted into other constructs. The interacting molecules, isolated by the methods described above, can be tested to see if the interaction is modulated when the primary binder or the amplifier binds its specific ligand. Another approach is to computationally determine the structure of an associated molecule that is designed to hide an epitope on a binder. Associated molecules can be designed as conjugates or as part of a peptide linked to or inserted at the carboxyl terminus of a whole antibody, Fab, scFv or diabody or any type of binder cited above. The purpose of an associated molecule is to modulate an epitope that is used to amplify a signal to detect a biomarker in a sample. If the primary binder, linker and amplifiers are structurally nucleic acids, nucleic acid associated molecules that modulate conformational epitopes are also possible.

Construction of a Primary Binder and an Amplifier Using a Combination of Naturally Occurring, Inserted or conjugated Epitopes or Associated Molecules

The foregoing embodiments of the present technology for the selection of binders have been presented for the purpose of illustration. They are not intended to be exhaustive or to limit the invention to the examples disclosed. There are many other possible ways to construct binders contemplated in this invention using a combination of naturally occurring, inserted and conjugated epitopes or associated molecules.

Other possibilities include, for example,

If a primary binder or secondary binder with one naturally occurring epitope has been isolated, the second epitope can be generated by

Creating random variance within the molecule

Splicing in a second naturally occurring epitope

Inserting an epitope

Making a conjugate

Isolating an associated binder

If two naturally occurring epitopes have been isolated but they are on different primary binders or secondary binders

Splice the two epitopes into one molecule

If no naturally occurring epitopes are identified by panning libraries

Create random variance within the binder

Insert one or more epitopes

Make multiple conjugates

Make a conjugate in combination with an inserted epitope

Isolate associated binders

Generate any combination of a naturally occurring epitope, an insert, a conjugate or an associated molecule

Design epitopes through computational methods

Oxidize residues or alter residues to create epitopes

The embodiments presented were chosen to illustrate to one skilled in the art examples of multiple ways to create reagents that meet the specifications of embodiments of the present technology.

Detection of Nucleic Acid Biomarkers

Embodiments of the present technology can be formatted to detect nucleic acids or any biomarker that has a suitable primary binder.

The detection of nucleic acid targets has an important role in clinical diagnostic and bio-terrorism. Although there are several target amplification technologies that can detect attomolar levels of a target, these technologies require several hours of processing time to amplify the target to a detectable level. A technology that produces a faster time to result would benefit many applications.

The detection of a nucleic acid target can be formatted in several possible ways, including, but not limited to, the following four techniques:

1. The primary binder, secondary binder, linker and amplifier or amplifiers are structurally proteins. The primary binder or linker is developed to have affinity and specificity for a sequence within the nucleic acid under assay. That primary binder, upon binding the nucleic acid under assay, produces a conformational epitope that is recognized by a specific secondary binder, linker, amplifier or amplifiers as described above.

2. The primary binder, secondary binder, linker, amplifier or amplifiers are structurally proteins. The primary binder is developed to have affinity and specificity for a conformational epitope that is created when the nucleic acid under assay binds a nucleic acid probe that is specific for the target and, in the process of binding the target, the probe produces an epitope that is recognized by the primary binder. The primary binder, upon binding the nucleic acid epitope, produces a conformational epitope that is recognized by a specific secondary binder, linker, amplifier or amplifiers as described above.

3. The primary binder, secondary binder, linker, amplifier or amplifiers are nucleic acids. The primary binder is selected to recognize a nucleic acid target or a probe that interacts with the target, as above, and in so doing produces a conformational epitope that is recognized by a secondary binder, linker, amplifier or amplifiers that produce conformational epitopes. All reagents in this format are nucleic acids.

4. The primary binder is a nucleic acid and the secondary binder, linker, amplifier or amplifiers are structurally proteins. The nucleic acid primary binder is developed to have affinity and specificity for the nucleic acid target and produce a conformational epitope that is recognized by a specific secondary binder, linker, amplifier or amplifiers.

Nucleic Acid System Advantages

Although current target amplification systems have exquisite sensitivity they require the use of enzymes and in some cases thermal cycling that adds cost and complexity to the system. In addition, these technologies require that nucleic acid extraction and amplification are performed as separate steps. In order to expose genomic DNA, cells must be lysed, DNA binding proteins removed and the DNA strands separated. This is usually accomplished by the addition of detergents, chaotropic reagents and heat. The chaotropic reagents and detergents are used to denature proteins and therefore are incompatible with enzymatic activity. As a consequence, nucleic acid extraction and amplification must be performed as separate steps. In addition, samples may contain enzyme inhibitors that need to be removed before amplification. The present technology provides a non-enzymatic nucleic acid amplification system that uses only nucleic acid primary binders, secondary binders, linkers and amplifiers offers the potential to integrate sample preparation and amplification into a single reaction process.

Formats—The following sections give detailed information on the various formats of the present technology, including but not limited to, amplification, FRET, FRET Linear amplification, other assay formats, viral detection, sample preparation, protein isolation, multiplexing and the like.

Performance Specifications of Secondary Binders

A mathematical model of the amplification reaction was developed to predict reaction conditions and detection parameters to achieve a rapid ultra sensitive result. The model teaches one schooled in the art, reaction conditions that will give the desired result.

### 1. Kinetics of Amplification Reaction

Association reaction kinetics between two entities can be described by the following equation:

$$A + B \begin{matrix}{\to }^{ka} \\ {←}_{kd}\end{matrix} AB \\ \frac{d [ AB ]}{d t} = {k}_{a} * [ A ] * [ B ] - {k}_{d} * [ AB ] \qquad ( Eq 1 )$$

Typical values for ka of Analyte-PB, PB-AMP and AMP-AMP binding reactions are 10⁵ to 10⁶ M⁻¹s⁻¹, typical kd are 10⁻³ to 10⁻⁴ s⁻¹.

If there were no limitations with respect to space, reagent supply, dissociation and diffusion, the molecular weight of an aggregate as described in this patent would increase exponentially with time, i.e.,

MW(t)=MW(AMP)2^{t·k}^{ₐ}^{·c(AMP)} (Eq 2)

Where MW(AMP) and c(AMP) are the molecular weight and the concentration of the amplifier, respectively. In reality, however, two restrictions have to be considered in order to obtain an accurate prediction of aggregate growth.

(i) Spatial Restrictions

The time it takes to add one additional layer of AMP (t_{iayer}) is given by Eq 3.

t_{Iayer}=1/(ka*c(AMP)) (Eq 3)

The radius of the aggregate (r_{aggregate}) as a function of time is therefore approximately:

r_{aggregate}≈r(PB)+2*r(AMP)*t*ka*c(AMP) (Eq 4)

If we furthermore assume that the packing fraction of AMP in the aggregate is Φ, Eq 5

V(AMP)*2^{t·k}^{ₐ}^{·c(AMP)}/Φ≦4π/3*r_{aggregate}³ (Eq 5)

must be fulfilled due to space requirements. V(AMP) is the volume of AMP. For typical values of V(AMP) and r(AMP) this inequality is true for r_{aggregate}=100 to 200 nm, N_{layer}=10 to 20, and MW/MW(AMP)=10⁴ to 10⁶.

This corresponds to the very beginning of the amplification reaction. During most of the reaction, the fastest possible reaction controlled (see Section (ii)) growth rate of the radius of the aggregate is therefore given by (Eq 6).

$$\frac{d r ( aggregate )}{d t} \approx ka * c ( AMP ) * 2 * r ( AMP ) \qquad ( Eq 6 )$$

Whereas exponential growth according to Eq 2 would lead to a growth rate described by Eq 7

$$\frac{d r ( aggregate )}{d t} = const * {2}^{t \cdot {k}_{a} \cdot c ( AMP ) / 3} \qquad ( Eq 7 )$$

which would imply that the radial growth rate increases to infinity which is clearly unphysical. (const is a constant which can be calculated on the basis of the geometrical properties of AMP). Therefore, Eq. 7 only applies to the very beginning of the amplification reaction. Afterwards, Eq. 6 applies.

### (ii) Limits of Diffusion

In addition to the limitation discussed in Section (i), a second important factor limits the growth rate. Implicit in Eq (1) is that all species are distributed homogeneously in the solution. A growing particle as described herein, however, uses a large number of AMP in order to grow, particularly as the particle grows larger. From Eq 6 it can be easily derived that the number of AMP molecules (n(AMP)) incorporated by the growing aggregate per unit time is given by Eq 8 and grows with the square of the aggregate radius.

$$\frac{d n ( AMP )}{d t} \approx 6 Φ ka * c ( AMP ) / {r ( AMP )}^{2} * {r ( aggregate )}^{2} \qquad ( Eq 8 )$$

This increasing consumption of AMP particles by the growing aggregate will change the mechanism of reaction from reaction controlled (as described by Eq 1) to diffusion controlled. It can be shown by calculations and computer simulation experiments that in the diffusion controlled region of the growth of an aggregate as described above, addition of AMP molecules to the aggregate can be described by Eq 9.

$$\frac{d n ( AMP )}{d t} = 4 π {rN}_{A} c ( AMP ) * 1000 l \text{/} {m}^{3} * D \qquad ( Eq 9 )$$

The transition from reaction controlled to diffusion controlled therefore occurs when Eq 10 is fulfilled:

6Φka/r(AMP)²*r(aggregate)²>4πr N_{A}*1000 l/m³*D (Eq 10)

Based on the above equations, the kinetics of the association reaction can be calculated and favorable reaction conditions can be predicted.

Dissociation of the aggregate will not play a significant role as it is very slow, and even though some bonds in the aggregate may break during its growth phase, they will immediately re-form due to the spatial proximity of the two partners.

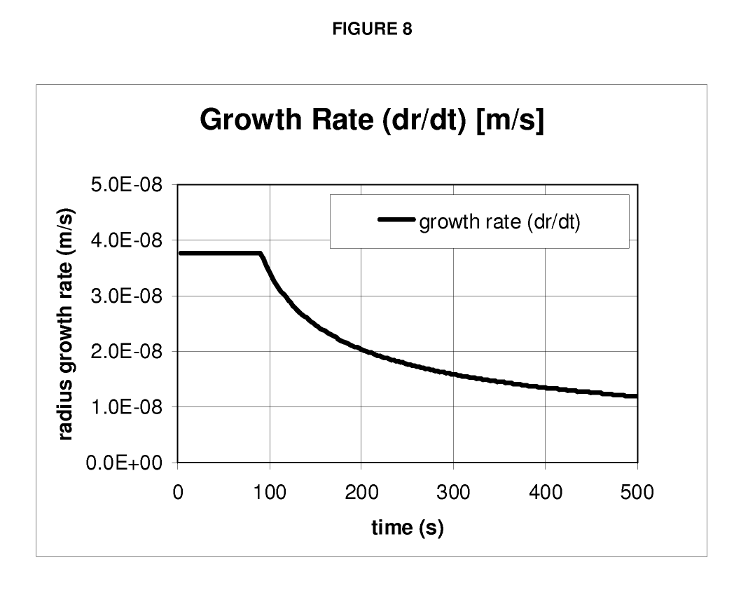

FIG. 8 predicts the growth rate for a reaction with two different amplifiers when the initial amplifier concentrations are at about 10 uM and the amplifier association rate constants are about 5E5(M⁻¹ s⁻¹). The rate at which the aggregate forms is initially limited by reaction rate but becomes diffusion limited around about 100 seconds into the reaction. This demonstrates that there is a fast growth rate, even in diffusion controlled limit.

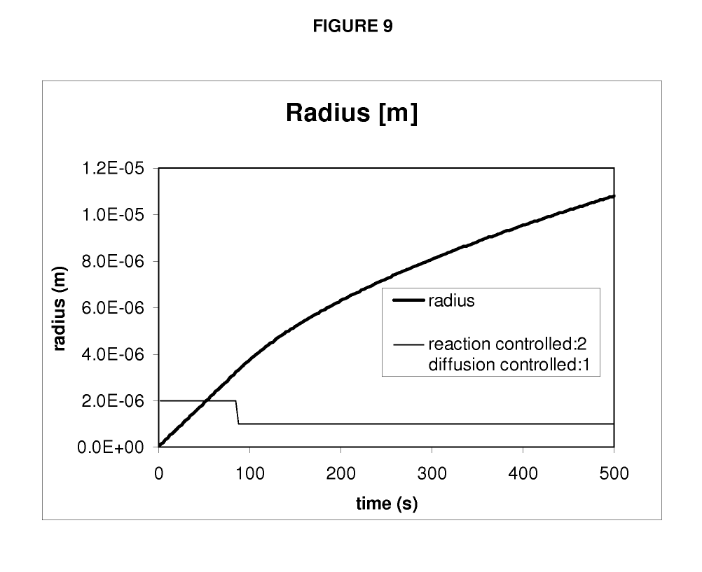

FIG. 9 predicts the aggregate radius with time with two different amplifiers when the initial amplifier concentrations are at about 10 uM and the amplifier association rate constants are about 5E5(M⁻¹ s⁻¹). At approximately 300 seconds into the reaction, the aggregate radius reaches about 8 um.

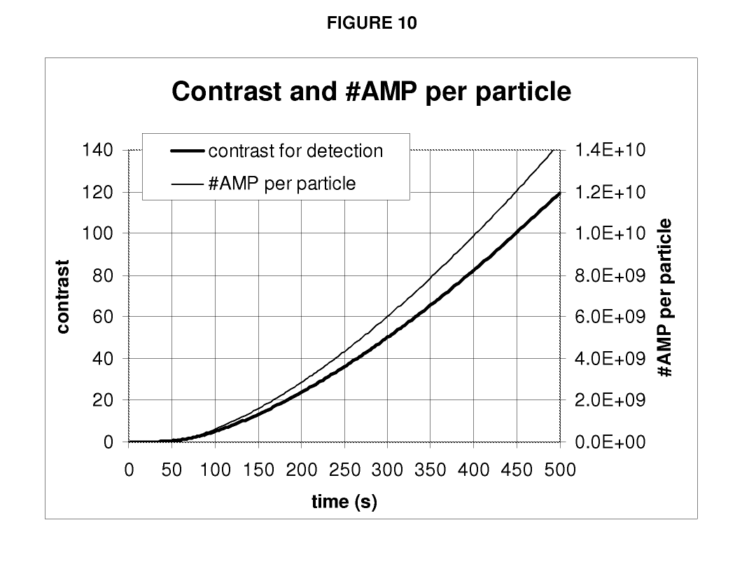

FIG. 10 predicts the contrast detected by the CCD (see next section) with time of reaction when the initial amplifier concentrations are at about 10 uM, the amplifier association rate constants are about 5E5(M⁻¹ s⁻¹) and the reaction has a volume of about 200 ul and the optical system has a about 2E7 pixel CCD camera. At approximately 300 seconds into the reaction, an aggregate reaches a contrast of ˜50.

FIG. 10 also predicts the number of amplifier molecules in the aggregate with time when the initial amplifier concentrations are at about 10 uM and the amplifier association rate constants are about 5E5(M⁻¹ s⁻¹). At approximately 300 seconds into the reaction the aggregate contains about 6E9 amplifier molecules.

### 2. Contrast for Detection

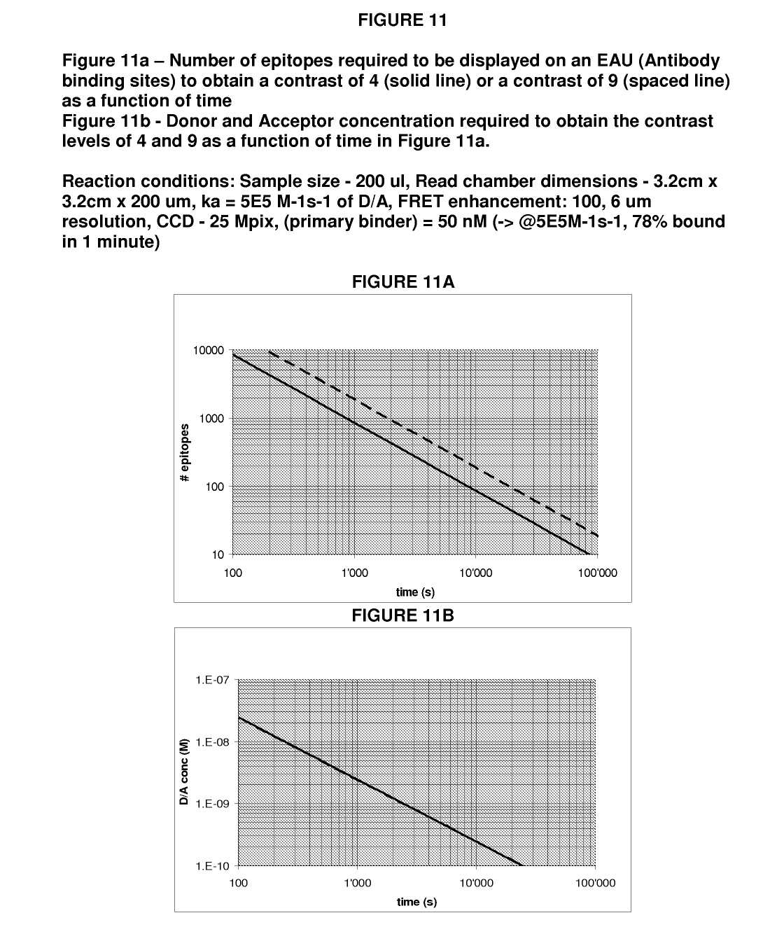

If each AMP is labeled (e.g., with a fluorophore), and the assay volume is illuminated with a light source suitable to excite the fluorophore and observed with a position sensitive detector (e.g., a CCD camera) as illustrated in FIG. 11, the aggregates will be visible as bright spots if they are large enough for detection.

The contrast for detection (CD) observed for an aggregate of a certain size can be calculated. If we assume that the volume of the assay is imaged in a CCD camera on Npix pixels, then the fluorescence background intensity (I_{background}) due to the unbound AMP is given by Eq 11, assuming that the majority of AMP is not bound to growing particles. The optical system has to be chosen is such a-way that the resolution is limited by Npix and not by the optical resolution of the imaging lens.

I_{background}=C1*Vassay*c(AMP)*10⁻⁶ ul/l*N_{A}/Nₚᵢₓ (Eq 11)

C1 is a constant which depends on properties of the detection system (such as geometry of the detection system, sensitivity of the CCD, intensity and type of light source, optics, etc.) and the assay reagents (type, concentration of fluorophore, optical properties of solution, etc.).

The signal intensity of an aggregate (I_{signal}) is given by Eq. 12 if the aggregate is imaged in one pixel of the CCD camera. Should the aggregate lie at the border of two or, in the worst case, four pixels, the signal intensity of the aggregate could be up to four times lower.

I_{signal}=C1*N_{aggregate} (Eq 12)

N_{aggregate} is the number of AMP molecules in an aggregate and can be calculated using Eqs 8 to 11.

The contrast for detection is therefore:

CD=I_{signal}/I_{background}=N_{aggregate}/(Vassay*c(AMP)*10⁻⁶ ul/l*N_{A}/Nₚᵢₓ) Eq 13)

assuming the aggregate lies within one pixel. As stated above, it could be up to 4 times lower if the aggregate lies precisely at the intersection of four pixels. It is proportional to the number of pixels and inversely proportional to the assay volume. The dependence on the amplifier concentration is more complicated since N_{aggregate} is also a function of c(AMP).

Other sources of background, such as fluorescence of impurities, signal of scattered excitation light not absorbed by the filter, electronic noise, etc. are not considered in I_{background}, i.e. I_{background} is an ideal value for optimal instrument set-up.

Other detection techniques such as confocal microscopy and evanescent wave illumination can also be used.

The label may also be an entity capable of chemiluminescence and the light emission can be induced by triggering the chemiluminescent reaction. Alternatively, the label may be a dye of appropriate intensity (molar extinction coefficient).

### Amplification Format

Mathematical modeling of the amplification reaction predicts that a cluster or aggregate of labeled amplifier molecules will form in solution and generate a signal above background in time. The assay reagent and optical system parameters that drive time to result include: primary binder and labeled amplifier or amplifiers concentration, forward rate constant of the primary binder and labeled amplifier or amplifiers, CCD pixel number and sample volume.

Table 2 summarizes the time to contrast 50 as a function of CCD pixel number and amplifier binders forward rate constant. As the pixel number increases the background per pixel decreases and the contrast improves. As the forward rate constant improves the time to result decreases because aggregate formation is more rapid.

TABLE 2

| CCD | Forward rate | Minutes to |
|---|---|---|
| Mega-pixels | ka E5 (M−1s−1) | Contrast 50 |
| 5 | 2 | 15.4 |
| 10 | 3 | 8.8 |
| 15 | 4 | 6.3 |
| 20 | 5 | 5.0 |
| 25 | 7 | 4.0 |
| 30 | 7 | 3.6 |

Constants
MW—165,000
2 amps
200 ul sample volume
Read Chamber (3.2 cm × 3.2 cm × 0.2 mm)
cAMP = 10 uM
c(primary binder) = 50 nM (−> @5E5(M−1s−1), 78% bound in 1 minute)

Table 3 summarizes the most preferred, more preferred and preferred ranges for key amplification reaction reagents and optical system imaging components for a 2 amplifier system.

TABLE 3

| | Most | | |
|---|---|---|---|
| | Preferred | More Preferred | Preferred |
| CCD (mpix) | about 12- | about 10-about | about 5- |
| | about 30 | 30 | about 30 |
| AMP - ka (E5M−1s−1) | about 2-about | about 1-about | about 0.1- |
| | 10 | 10 | about 10 |
| AMP - (uM) | about 1-about | about 0.5- | about 0.05- |
| | 100 | about 100 | about 100 |
| PB (nM) | about 1-about | about 0.1- | about 0.1- |
| | 100 | about 100 | about 1000 |
| Sample Volume (ul) | about 100- | about 50-about | about 10- |
| | about 300 | 300 | about 1000 |
| Chamber | about 0.1- | about 0.05- | about 0.01- |
| Dimensions, depth | about 0.3 mm | about 0.5 mm | about 1.0 mm |
| Chamber Dimensions, | about 3.2 cm × | about 4 cm × | about 4 cm × |
| length × width | about 3.2 cm | about 4 cm | about 4 cm |
| | | to about 2 cm × | to about 1 cm × |
| | | about 2 cm | about 5 cm |
| PB - Type | Mab, Fab | Mab, Fab | Mab, Fab, |
| | | | ScFv |
| AMP - Type | Mab, Fab | Mab, Fab | Mab, |
| | | | Fab, ScFv |

The amplification format produces rapid results with single cluster sensitivity when the CCD is >5 mpix, AMP ka is >2E5(M-1s-1), AMP concentration is ≈10 uM and the sample volume is ≈200 ul. (See, Tables 2 and 3)

Dynamic range of the assay—The use of a 20 mpix CCD can permit about 1 million amplification centers to be resolved which will permit quantification of an analyte between 0 and ˜1 million molecules. With time, inexpensive CCDs in the range of 25-30 mpix should be available which should permit resolution of several million amplification centers.

Reagent concentrations—Table 3 shows the reagent ranges for the amplification format. When a primary binder is present around about 50 nM and the labeled amplifiers are present around about 10 uM a rapid time to contrast 50 is achieved, at about 5 minutes, using 2 amplifiers each having a ka of about 5E5(M-1s-1) and a 20 mpix CCD. See, Table 2 and Table 3.

Sample volume—A sample volume in the range of about 100−about 300 ul is most preferred. That volume fills a chamber with the dimensions of about 3.2 cm×about 3.2 cm×(about 100−about 300 um) which is most preferred.

### Advantages of the Amplification Format

Some advantages of the amplification format include, but are not limited to,

1) when an antibody primary binder binds its specific analyte and exposes a conformational epitope it is acting as a transducer that converts a binding event into a signal generating event. The transduction process used in this invention is the same or similar to the transduction process used during a normal immune response. It is therefore anticipated that many existing antibodies will be able to initiate the amplification reaction;

2) the reaction is self sustaining. There are no enzymes required to produce a result;

3) the reaction is homogeneous. There is no need to separate the product from the reactants in order to read the result;

4)The method is simple; add sample to reagent, incubate and read;

5) The reaction is rapid; results are obtained in minutes; and

6) The primary binder is not labeled. Therefore it can be added at high concentrations to drive the rate of reaction without increasing background as in conventional formats.

In the most preferred embodiment, the present technology comprises
Sample, for example, serum or plasma, and reagent are added together to produce a final volume between about 100 and about 300 ul, the primary binder is present at about 1−about 100 nM, the two amplifier binders are each present at 1-100 uM and the ka of the primary binder and amplifier binders is about 2E5-10E5 (M-1s-1).
The sample is placed in a chamber about 3.2 cm×about 3.2 cm×(about 0.1−about 0.3 mm).
The sample is imaged onto a CCD having 12 megapixels or greater.
An image is taken at t=0 to measure average pixel signal.
An image is taken at t=X to measure pixel signal.
System controls to include: no sample, 0 analyte, low level analyte, mid level analyte and high level analyte are run as required.
The primary binder and labeled amplifier molecules are Mabs or Fabs.
The conformational epitopes on the primary binder are the C1Q and FCgR1 binding sites.
The specificity of the labeled amplifier molecules are anti-C1Q binding site and anti-FcgR1 binding site.
The label is fluorescein.

Types of samples suited for the amplification format—In theory, any analyte at any concentration can be used in the amplification reaction. However, the samples that would benefit most from the amplification format's unique performance include, but are not limited to, for example:
Low level of proteins—cancer markers
Category B agents of terror—toxins
Category A agents of terror—organisms
Bacterial, fungal, viral (identification and quantification)

Additional techniques to improve performance
When background aggregates are found in the “no sample control”, these background aggregates must be subtracted from true sample aggregates. A possible cause of no sample aggregate formation may be denatured amplifier molecules that expose a hidden epitope that initiates the amplification reaction.
Extremely high analyte concentrations will consume all the amplifier molecules into tiny aggregates that will not produce a signal above background. This background, “numerous tiny aggregates”, will follow a Poisson distribution that is recognizably different from a “no sample” background. Samples that show this higher variability, Poisson distribution, will be identified, diluted and rerun.

Although it is desirable to maximize assay performance, for example, by producing results as fast as possible, by using a high concentration of reagents, by using amplifiers with the fast association rate constant and detectors with a large pixel number, a practical approach dictates that performance may be constrained by cost considerations. It is desirable therefore to balance the system to obtain the highest level of performance at a reasonable level of cost.

In addition, the present technology includes many methods to isolate or construct primary binders and amplifiers. In one embodiment, the amplification system contains an amplifier with two identical conformational epitopes. This is the most efficient system with the lowest manufacturing cost. Since a binder with two identical, non-overlapping, conformational epitopes in its superstructure may not be found in a library, the binder may need to be engineered using splicing and inserting techniques. There are many ways to engineer the construct and the exact method can be guided by structural, sequence and computational methods, techniques which are similar to those described by the following references (Lippow, Almagro, Carter, Glaser, Swers, and Patrick, incorporated by reference in their entirety). In a second embodiment, the amplification system uses two amplifiers with different conformational epitopes. This system is based on the isolation of a primary binder that has two secondary binders using a complex library. The engineering of the secondary binders into amplifiers using the superstructure of the primary binder is an established process. The mathematical model teaches that the use of two amplifiers requires that each amplifier be present at the same concentration to maintain the same rate of reaction. Since the effective concentration of amplifier is doubled in this case, background noise will increase by a factor of two and the CCD pixel number will need to be increased by a factor of two in order to obtain the same time to result. In some embodiments, the system has one amplifier which can be more efficient and cost effective, but more complex to engineer. In alternative embodiments, the system has two amplifiers which may be easier to engineer.

There are several conformational sites in the Fc portion of an antibody that have been extensively studied, e.g., the C1Q binding site and the FcgR1 binding site. Binders can be isolated that bind to these sites and engineered to meet the performance specification required of the amplification format described above. The term binder to the C1Q binding site or binder to the FcgR1 binding site in no way implies that the binder and the C1Q or FcgR1 share anything in common other than they compete for the same region of the antibody and interfere with binding to the site by the other binder.

Taken in whole, the methods to select binders described above, the binder performance specifications described above, the reaction conditions described above and the detection conditions described above teach one schooled in the art how to obtain single molecule detection of an analyte in a sample using an easy to use, rapid, homogeneous format. (The term single molecule is intended to include single molecules, molecular complexes, viruses, microorganisms or parts thereof.)

### FRET Format

Cellular analysis offers a special opportunity because the CUA or EUA may display repetitive epitopes which enable endogenous amplification that can be utilized to enhance detection. When multiple copies of a target molecule are displayed on the CUA or EUA the FRET format of the present technology may be used. Its unique design may enhance performance and solve problems that have limited cell based analysis in the past. For example, the FRET format may be useful when a cell based assay has high background due to non specific binding or cross reactivity or when a cell based assay lacks sensitivity.

### A.1. Kinetics of FRET System

In one embodiment of the FRET system, the analyte under assay (AUA) is combined with the primary binder (PB), one secondary binder labeled with a fluorescent donor (SBD) and one secondary binder labeled with a fluorescent acceptor (SBA). The AUA can consist, for example, of a cell or a virus with a certain number (NE) of a particular epitope (EPI) expressed on its surface.

If we assume that the concentrations of the analyte under assay, the secondary binder labeled with a fluorescent donor, and the secondary binder labeled with a fluorescent acceptor are [AUA], [SBD] and [SBA], respectively, the system can be described by the following kinetic equations, where the concentration of the epitope [EPI] is obviously [EPI]=NE*[AUA]:

$$\begin{matrix} EPI + PB \begin{matrix}{\to }^{ka ( PB )} \\ {←}_{kd ( PB )}\end{matrix} EPI * PB & ( Eq 14 a ) \\ EPI * PB + SBA \begin{matrix}{\to }^{ka ( SBA )} \\ {←}_{kd ( SBA )}\end{matrix} EPI * PB * SBA & ( Eq 14 b ) \\ EPI * PB + SBD \begin{matrix}{\to }^{ka ( SBD )} \\ {←}_{kd ( SBD )}\end{matrix} EPI * PB * SBD & ( Eq 14 c ) \\ EPI * PB * SBD + SBA \begin{matrix}{\to }^{ka ( SBA )} \\ {←}_{kd ( SBA )}\end{matrix} EPI * PB * SBA * SBD & ( Eq 14 d ) \\ EPI * PB * SBA + SBD \begin{matrix}{\to }^{ka ( SBD )} \\ {←}_{kd ( SBD )}\end{matrix} EPI * PB * SBA * SBD & ( Eq 14 e ) \\ The kinetics of Eq ( 14 a \text{-} e ) can be described by Eq ( 15 a \text{-} e ) . & \\ \frac{d [ EPI * PB ]}{d t} = {k}_{a} ( PB ) * [ EPI ] * [ PB ] - {k}_{d} ( PB ) * [ EPI * PB ] & ( Eq 15 a ) \\ \frac{d [ EPI * PB * SBA ]}{d t} = {k}_{a} ( SBA ) * [ EPI * PB ] * [ SBA ] - {k}_{d} ( SBA ) * [ EPI * PB * SBA ] & ( Eq 15 b ) \\ \frac{d [ EPI * PB * SBD ]}{d t} = {k}_{a} ( SBD ) * [ EPI * PB ] * [ SBD ] - {k}_{d} ( SBD ) * [ EPI * PB * SBD ] & ( Eq 15 c ) \\ \frac{d [ EPI * PB * SBA * SBD ]}{d t} = {k}_{a} ( SBA ) * [ EPI * PB * SBD ] * [ SBA ] - {k}_{d} ( SBA ) * [ EPI * PB * SBA * SBD ] & ( Eq 15 d ) \\ \frac{d [ EPI * PB * SBA * SBD ]}{d t} = {k}_{a} ( SBD ) * [ EPI * PB * SBA ] * [ SBD ] - {k}_{d} ( SBD ) * [ EPI * PB * SBA * SBD ] & ( Eq 15 e )\end{matrix}$$

Typical values for ka are 10⁵ to 10⁶ M⁻¹ s⁻¹. Typical values for kd are 10⁻³ to 10⁻⁴ s⁻¹.

Solving the system of equations (15a) to (15e) allows the calculation of [EPI*PB*SBA*SBD] etc. as a function of time.

### A.2. Contrast for Detection of FRET System

Fluorescence donor and fluorescence acceptor both have an absorption and emission spectrum. The absorption and emission spectra of the donor will be shifted to lower wavelengths than the absorption and emission spectra of the acceptor. If the donor (SBD) and acceptor (SBA) are sufficiently far apart, typically more than about 10 nm, which corresponds to a donor and acceptor concentration of less than 0.2 mM each, essentially no FRET signal will be observed. In such a case, if the fluorescence in a sample volume is excited at a short excitation wavelength (aₑₓ), where the absorption of the donor is high and the absorption of the acceptor is low, mainly the fluorescence of the donor will be excited. If we now observe the fluorescence signal at a long emission wavelength (λₑₘ) where the fluorescence intensity of the donor is low and the fluorescence intensity of the acceptor is high, we will only observe a very small fluorescence signal (I_{fluo,random}). This signal intensity is the “background” and is proportional to the concentrations of donor and acceptor, the ratio of acceptor to donor absorption at λₑₓ, and normalized (with respect to absorbed photons) donor to acceptor emission at λₑₘ.

If SBD and SBA are in close proximity, closer than the Förster distance which is in the order of 5 nm depending on the nature of A and D, Fluorescence Resonance Energy Transfer (FRET) will be observed. If we excite the donor fluorescence in such a case at λₓ, the donor will transfer the energy in a non-radiative mode to A, and A will fluoresce with its characteristic spectrum. Therefore, the fluorescence intensity at (λₑₘ), will be high. We define this fluorescence intensity as I_{fluo,close} when all donors and acceptors within the sample volume are at close distance with each other. The corresponding signal enhancement (FRET enhancement) is defined as the ratio I_{fluo,close} of and I_{fluo,random}.

Using the same optical setup as described above (FIG. 12), the fluorescent background can be calculated by Eq. 16 in analogy to Eq 11.

I_{background}=C2*Vassay*([SBA]+[EPI*PB*SBA]+α{[SBD]+[EPI*PB*SBD[})*10⁻⁶ ul/l*N_{A}/Nₚᵢₓ (Eq 16)

α is a constant and equal to the emission intensity of D divided by the emission intensity of A when D and A are far apart and excited at λₑₓ and observed at λₑₘ.

C2 is a constant which depends on properties of the detection system (such as geometry of the detection system, sensitivity of the CCD, intensity and type of light source, optics, etc.) and the assay reagents (type, concentration of fluorophore, optical properties of solution, etc.).

Other sources of background, such as fluorescence of impurities, signal of scattered excitation light not absorbed by the filter, electronic noise, etc. are not considered in I_{background}, i.e. I_{background} is an ideal value for optimal instrument set-up.

The signal intensity of one AUA (I_{signal}) is given by Eq. 17 if the AUA is imaged in one pixel of the CCD camera.

I_{signal}=C2*FRET enhancement*NE*{[EPI*PB*SBD*SBA]/EPI₀} (Eq 17)

where EPI₀=[EPI*PB*SBD*SBA]+[EPINEPI*PBNEPI*PB*SBDHEPI*PB*SBA]

The contrast for detection (CD) becomes then:

$$CD = {I}_{signal} {/}_{background} = FRET enhancement * NE * \{ [ EPI * PB * SBD * SBA ] / {EPI}_{0} \} / \{ Vassay * ( [ SBA ] + [ EPI * PB * SBA ] + α \{ [ SBD ] + [ EPI * PB * SBD ] \} ) ) * {10}^{- 6} ul \text{/} l * {N}_{A} / Npix \} \qquad ( Eq 18 )$$

[EPI*PB*SBD*SBA] etc. can be calculated using Eqs. 15a to 15e.

In order to optimize the contrast, [EPI*PB*SBD*SBA]/EPI₀] has to be optimized while keeping [SBA]+[EPI*PB*SBA]≈[SBA] at a minimum.

An extremely low SBA concentration would give an extremely long reaction time. Therefore, the aim is to choose the optimal SBA concentration for a given allowed reaction time. It can be shown that the optimal SBA and SBD concentrations for a given reaction time t₀ have to be chosen in such a way that after t₀ about 71% of the A sites of EPI*PB and 71% of the D sites of EPI*PB are occupied.

In FIG. 11 the results of such calculations are depicted. FIG. 11Bshows, e.g., that for an allowed reaction time of 1000 s, a concentration of 2.5 nM has to be chosen for SBA and SBD, and FIG. 11A shows that under these conditions an EUA with NE 850 will lead to a contrast of 4, while an EUA with NE≈1900 will lead to a contrast of 9.

The binding of PB to EPI is generally not a time critical step if [PB] is chosen as depicted in Table 1.

FIG. 11A predicts the time required to obtain a contrast level of 4 or 9 as a function of the number of epitopes displayed on the entity under assay using the FRET format. At 600 seconds an entity under assay displaying ˜1500 epitopes will generate a contrast of 4 on the CCD and an entity displaying ˜3000 epitopes will generate a contrast of 9 on the CCD.

FIG. 11B predicts the donor and acceptor concentration required to obtain the contrast level of 4 or 9 shown in 11A. To obtain the contrast levels of 4 and 9 at 600 seconds predicted in 11A the reaction will require a donor and acceptor concentration of 4 nM.

Table 1 summarizes the time to contrast 9 predictions for a FRET cluster as a function of CCD pixel number, donor and acceptor association rate constant, donor and acceptor concentration and number of epitopes displayed on the entity under assay. The time to contrast 9 decreases as the pixel number increases, the forward rate constant increases and the number of binding sites on the EAU increases. The data predict that an aggregate of contrast 9 will be formed in ˜2.5 minutes when the entity displays 10,000 epitopes using a 25 megapixel CCD and a donor and acceptor pair with a ka of 7E5(M⁻¹ s⁻¹) at a concentration of SBD and SBA of 1.2E-8 M.

Mathematical modeling of the FRET reaction predicts that a cluster or aggregate of FRET pairs will form in solution and generate a signal above background in time. The assay reagent and optical system parameters that drive time to result include: concentration of the primary binder, FRET labeled Donor and Acceptor binder and FRET partner; forward rate constant of the primary binder, FRET labeled Donor and Acceptor binder, FRET partner, the efficiency of the FRET reaction, CCD pixel number and sample volume.

Table 4 summarizes the most preferred, more preferred and preferred ranges for key reaction reagents and optical system imaging components for the FRET system. The data show that the number of primary binder binding sites on the EUA and the CCD pixel number drive the concentration of the donor and acceptor used in a specific assay. As the EAU becomes smaller or the number of displayed epitopes decreases, the signal generated by the EAU becomes smaller and therefore the concentration of reagents must be decreased to lower background to optimize results. As the pixel number increases the background is spread over more pixels and the concentration of reagents can be increased to optimize results.

TABLE 4
Table 4

| | Most | | |
|---|---|---|---|
| | Preferred | More Preferred | Preferred |
| CCD (mpix) | about 12- | about 10-about | about 5- |
| | about 30 | 30 | about 30 |
| ka E5(M−1s−1) | about 2-about | about 1-about | about 0.1- |
| | 10 | 10 | about 10 |
| PB (nM) | about 1-about | about 0.1-about | about 0.1- |
| | 100 | 100 | about 1000 |
| Sample Volume (ul) | about 100- | about 50-about | about 10- |
| | about 300 | 500 | about 1000 |
| D/A (M)/NE = | about 3.7E−11- | about 1.8E−11- | about 4.6E−12- |
| 100/CD = 9 | about 2.8E−10 | about 5.5E−10 | about 2.8E−9 |
| D/A (M)/NE = | about 3.7E−10- | about 1.8E−10- | about 4.6E−11- |
| 1,000/CD = 9 | about 2.8E−9 | about 5.5E−9 | about 2.8E−8 |
| D/A (M)/NE = | about 3.7E−9- | about 1.8E−9- | about 4.6E−10- |
| 10,000/CD = 9 | about 2.8E−8 | about 5.5E−8 | about 2.8E−7 |
| D/A (M)/NE = | about 3.7E−8- | about 1.8E−8- | about 4.6E−9- |
| 100,000/CD = 9 | about 2.8E−7 | about 5.5E−7 | about 2.8E−6 |
| Chamber Dimensions, | about 0.1- | about 0.05- | about 0.01- |
| depth | about 0.3 mm | about 0.5 mm | about 1.0 mm |
| Chamber Dimensions, | about 3.2 cm × | 4 cm × 4 cm | 4 cm × 4 cm |
| length × width | about 3.2 cm | to 2 cm × 2 cm | to 1 cm × 5 cm |
| PB - Type | Mab, Fab | Mab, Fab | Mab, Fab, |
| | | | ScFv |
| D/A - Type | Mab, Fab | Mab, Fab | Mab, Fab, |
| | | | ScFv |

The D/A concentration is adjusted depending on the CCD pixel number and sample volume. For example, box (more preferred / NE=100, sample volume=200 ul) when the CCD is 15 mpix the D/A(M) is about 6.9E-11 when the CCD is 30 mpix the D/A(M) is about 1.4E-10. [D/A(M) is the concentration of SBD and SBA, NE is number of primary binder molecules bound to EUA, CD is contrast obtained on CCD].

The FRET format produces rapid results (less than 1 hour) with single cluster sensitivity when the CCD is >5 mpix, the ka of the primary binder and FRET pair are >2E5(M-1s-1), primary binder concentration is ˜50 nM, the FRET pair concentration is >2.3E-9M, the EUA has ˜10,000 binding sites and the sample volume is ˜200 ul. See, Table 1 and Table 4.

Dynamic Range of the Assay—In the present technology, the use of a 20 mpix CCD should permit ˜1 million EUAs to be resolved which will permit quantification of an EUA between 0 and ˜1 million entities. With time, inexpensive CCDs in the range of 25-30 mpix should be available which should permit resolution of several million amplification entities.

Reagent Concentrations—Table 4 shows the reagent ranges for the FRET format. For example, when the EUA presents ˜10,000 binding sites the primary binder is present at around 50 nM and the labeled FRET pair is present around 1.2E-8(M) a contrast of 9 is obtained in ˜2-3 minutes using a FRET pair with a ka of 7E5(M-1s-1) and a 25 mpix CCD. (Table 1 and Table 4).

Sample Volume—A sample volume in the range of about 100−about 300 ul is most preferred. That volume fills a chamber with the dimensions of about 3.2 cm×about 3.2 cm×(about 100−about 300 um) which is most preferred.

Epitope Range—The FRET format is useful for entities that can bind >500 primary binders.

In the Most Preferred Embodiment
Sample, for example, serum or plasma, and reagent are added together to produce a final volume between about 100 and about 300 ul, the primary binder is present between about 1−about 100 nM, the D/A are each present between about 3.7E-11−about 2.8E-7M (depending on number of epitopes displayed on EAU and CCD pixel number) and the ka of the primary binder and D/A is between about 2E5-10E5 (M-1s-1).
The sample is placed in a chamber about 3.2 cm×about 3.2 cm×(about 0.1−about 0.3 mm).
An image is taken at t=0 to measure average pixel signal.
An image is taken at t=X to measure pixel signal.
System controls of: no sample, 0 EUA, low level EUA, mid level EUA and high level EUA are run as required.
The primary binder is a Mab or Fab.
FRET pairs are Fabs or Mabs.
The conformational epitopes on the primary binder are, for example, the C1Q and FCgR1 binding sites.
The specificity of the FRET pair is, for example, anti-C1 Q binding site and anti-FcgR1 binding site.
The D/A pair are, for example, Alexa 594/Alexa 610 or Alexa 594/Alexa 633.

The following uses specific conformational epitopes known to one in the art, however, any conformational epitopes that will properly align a FRET pair can be used.

As stated above, there are several known conformational epitopes in the superstructure of antibodies, the C1Q binding site and the FcgR1 binding site. These sites are physically located close to each other and reside in the Fc region on an antibody. The C1Q binding site is in the CH2 domain and the FcgR1 binding site is at the junction of the CH2 and CH3 domain. It is estimated that the distance between the sites is <2 nm. These tandem conformational epitopes can be used as docking sites for a FRET pair used to generate a detection signal (FIG. 6B). A mouse monoclonal may be used as the primary binder to specifically identify the presence of the CUA or EUA in the sample. Upon binding its specific epitope, the primary antibody will undergo conformational changes exposing the C1Q and FcgR1 binding sites. Secondary binders that recognize these conformational sites and are labeled with a donor and acceptor FRET pair will bind to the sites which will align the molecules in close proximity. Excitation of the donor molecule with the appropriate wave length will produce a FRET signal identifying the presence of the EUA.

Types of Samples Suited for the FRET Format—In theory any EUA that binds >500 primary binder molecules can be used in the FRET format, however, the time to result increases as the number of available binding sites decrease on the EAU. Samples that display ˜10,000 binding sites will produce rapid results.

Typical EUAs for this format include, but are not limited to, for example: pathogens like bacteria and fungi, cells, spores, viruses and the like.

The following FRET pairs are examples of donor and acceptor molecules that might be useful in the FRET format. The dyes shown below are commercially available. The excitation and emission wavelength differs among pairs. A pair is chosen based on application, coupling chemistry and performance in the final assay.
Cy3-Cy5
Alexa 488—Alexa 555
Alexa 488—Cy3
FITC—Rhodamine
IAEDANS—Fluorescein
EDANS—DABCYL
Cy5-Cy5.5
Cy5-Cy7Q
FAM—TAM RA
   Advantages of the FRET system

Some advantages of using a FRET system with embodiments of the present technology, include:
Sufficient contrast for detection is obtained in minutes.
The method is simple—add sample to reagent, incubate and read.
The reaction is homogeneous—there is no need to separate the product from the reactants in order to read the result.
The signal generating system has multi-assay utility. Antibodies from a specific species and a specific subclass share the same constant domains CL, CH1-CH2-CH3; therefore, when a FRET pair is identified for an antibody subclass, that pair in theory can be used with any antibody from that subclass that exposes the same epitopes. The hidden conformational epitope or epitopes that are exposed upon binding of the primary binder to its specific EUA act as transducers that convert a binding event into a signal generating event. The transduction system is common to all antibodies of the subclass.
FRET signal generation using conformational epitopes has advantages in specificity and sensitivity when compared to systems that directly label the primary binder. The primary binder is not labeled therefore it can be added at high concentrations to drive the rate of reaction without increasing background as in conventional formats. Non-specific binding due to weak molecular interactions is significantly reduced using the FRET format because the primary binder is not labeled. In a classical heterogeneous immunoassay, non-specific binding limits sensitivity because it raises background noise. The non-specific binding is caused when the labeled primary binder binds to other molecules bound to solid phase supports through weak forces which include hydrophobic, electrostatic, ionic, Van der Waals and hydrogen binding. The molecules interact at multiple points producing sufficient binding avidity to be “sticky”. The use of conformational epitopes greatly reduces problems of non-specificity because the primary binder in the FRET assay is not labeled and the primary binder needs to be bound to its specific antigen to expose conformational epitopes for FRET pair alignment. Background in the FRET system is a function of donor and acceptor concentration up to ˜1 uM and low level system contamination. The probability of non-specific FRET signal generation is lower than the probability of a labeled primary binder sticking to a solid phase support. Therefore, specificity improves and the background is lowered which permits the minimum detectable quantity to be lowered, which improves sensitivity. A FRET assay designed with stable epitopes as opposed to the conformation specific epitopes of the present technology would show no improvement because its background would be a function of the “sticky” primary binder and the concentration of the D and A, and also require much higher concentrations of D and A since they would have to match the concentration of the primary binder.
The FRET format will greatly reduce background non-specific binding because the Donor and Acceptor will only bind to a properly oriented primary binder which provides advantages over known methods of tumor cell detection which have non-specific binding issues.
It is possible that a primary antibody will show some binding affinity for molecules other than its specific antigen. Examples include; members of the same protein family, proteins that share a common domain and molecules that by chance have a similar shape. This cross reactivity is usually of much lower affinity with a much faster off rate (kd) than the interaction with the specific antigen. These interactions however raise the background of an assay and reduce analytical sensitivity. The use of conformational epitopes to generate a signal will help reduce this source of background, since these interactions are unstable and have a high off rate. The probability that a donor and acceptor FRET pair will properly orient due to low affinity binding to other antigens, therefore, is greatly reduced.
The use of conformational epitopes in the FRET format incorporates multiple layers of specificity control for EUA identification and quantification. Generation of the FRET signal is dependent on 3 essential and sequential interactions. 1. The specific antigen must be present and the primary binder must be bound to the specific antigen with high affinity. 2. The interaction of the antigen and primary binder must produce at least one conformational epitope. 3. The FRET pair has specificity for the conformational epitopes and must be aligned by the conformational epitopes to generate a signal.
The FRET format increases sensitivity by way of background reduction.

### FRET Linear Amplification Format

The number of epitopes presented by an EUA, primary binding sites, may range from 1->100,000. This wide range of binding sites creates both clear applications for the various technology formats of this invention and in other cases multiple formats may be considered. If one assumes that a rapid result is always desirable, then when an EUA has <500 binding sites the amplification format is the format of choice; when the EUA has >10,000 binding sites the FRET format is the format of choice; and when the EUA has between 500 and 10,000 the FRET linear amplification format is a possibility. The linear amplification relies on a primary binder or a linker that produces 3 conformational epitopes. Two of the sites are used to bind and align a FRET pair and the 3^{rd} site binds a secondary binder that is specific for the 3^{rd} conformational epitope on the primary binder. Upon binding the primary binder the secondary binder produces the same 3 conformational epitopes. The reaction continues without further intervention. When the linear amplifier is present at about 100 nM and has a forward rate constant of about 5E5(M-1s-1) the signal on the EUA will increase by a factor of 10 in ˜3 minutes. This format can be used to extend the lower range of the FRET format or to decrease time to result. When a stable epitope on a primary binder is used to generate a FRET signal, the binder that binds that stable epitope must be increased in concentration to the same level as the primary binder to assure that most sites become rapidly occupied, which increases background and decreases the time to contrast. The linear amplifier will help to reduce the time to contrast in this case. The FRET linear amplification format may also be useful as a method to directly detect the presence of virus particles in a sample.

### Advantages of the Linear FRET Format

Some advantages of using a linear FRET format with the present technology, include, but are not limited to: the linear FRET format useful when the signal needs to be amplified by a factor of 10-50; once developed the reagents can be used with any primary binder or linker that produces the appropriate conformational epitope; and the linear amplification format is controlled by the same layers of specificity as the FRET format

### FRET Discussion

In a preferred embodiment of the present invention, the FRET format is the method of choice when the EUA displays a high number of antigens or epitopes that can be used as binding sites for primary binders. It is possible that the EUA may have some or all of the binding sites occupied by antibodies produced by the host's immune system. A sample preparation step (see sample preparation section below) may be required to remove the endogenous binders before initiating the FRET format. The use of conformational epitopes to provide the docking sites for the FRET pair provides a unique way to improve assay specificity. In the FRET format, the primary binder is not labeled which is an advantage. The primary binder must be bound with high affinity to its specific target to produce conformational epitopes and the FRET pair must be aligned by the conformational epitopes to produce a specific signal. The format provides single cell sensitivity. The surface of the EUA becomes coated with primary binder and the FRET pair which produces a cluster of signaling molecules. With time the signal generated by the EUA becomes larger than background. Each signaling cluster is produced by one EUA. The format produces results within minutes. Further, the format is easy to use. The reaction is initiated by adding a single reagent to the sample. No washes or separation steps are required. Cluster detection takes place with simple and inexpensive instrumentation.

### Other Assay Formats

### Amplification Chamber Format

One embodiment of the present technology dispenses an appropriate volume of sample to be tested and a volume of reagent containing primary binder and labeled amplifier into a chamber and the mixture is incubated for an appropriate period of time. The resulting aggregates are read directly from the incubation chamber, if appropriate, or the reaction volume is transferred to a read chamber with appropriate dimensions and the contents of the chamber illuminated with a laser or other suitable light source and emitted photons imaged using a CCD camera or other suitable detector and the number of aggregates determined. One embodiment is a homogeneous format requiring no washes or separations.

In this embodiment, the reason that it is possible to detect single molecules in a homogeneous format without separating background molecules is based on two factors:
Spreading the background over X million pixels reduces the background in any one pixel by X million fold.
The chain reaction forms an aggregate of labeled amplifier molecules that is many times brighter than signaling systems used in the past.

Neither spreading the background over many pixels nor forming a much brighter aggregate of amplifier molecules is generally sufficient by itself. It is the combination of the two that permits detection of aggregates on the CCD without separation.

### Amplification Filter Format

Alternatively, an appropriate volume of sample to be tested and an appropriate volume of reagent containing primary binder and labeled amplifier are dispensed into a chamber and the mixture incubated for an appropriate period of time. The resulting aggregates are captured on a porous filter, ˜1 cm×1 cm with a pore size of 1 um, of low background and washed if appropriate. Other filters having a surface dimension between 0.5×0.5 and 3.2×3.2 cm squared in a device having a depth between 0.5 and 4 cm can also be used. The volume of solution containing the aggregates or the complex that is analyzed is typically between 0.1 and 10 ml.

The filtered material is illuminated with a suitable light source. Emitted photons are imaged using a filter and CCD camera or other suitable detector and the number of aggregates determined. In this embodiment, when aggregates are filtered onto the porous membrane the background is reduced because labeled amplifier molecules that are not incorporated into aggregates are small enough to pass through the membrane. Once the background is reduced, aggregates become visible when illuminated with an appropriate light source and emitted photons imaged using a CCD camera. This filter format is advantageous when large sample volumes are being interrogated. Determining the number of aggregates in the reaction and knowing the sample volume tested and any dilution made to the sample tested, the analyst is able to calculate the concentration or total number of molecules of the analyte under assay in the sample. One embodiment assures the quality of the answer through the use of standards with established analyte levels, the creation of a calibration curve, or other techniques typically used by analysts.

Examples of additional formats include, but are not limited to, for example, the following:

### Amplification Reaction Heterogeneous Format I

An appropriate volume of sample to be tested and an appropriate volume of reagent containing primary binder and amplifier or amplifiers are dispensed into a chamber and incubated for an appropriate period of time. The primary binder binds the analyte which initiates the amplification reaction. Aggregates are captured on a solid phase by way of an immobilized binder that recognizes a conformational epitope on amplifier molecules in the aggregate, washed, reacted with a labeled binder specific for the aggregate, washed and detected. The binder can be labeled with any signal generating molecule, for example a fluorescent molecule for detection. This format produces a standard forward sandwich immunoassay with detection integrating the entire bulk solution. This format is not a single molecule detection technology but requires a standard curve to determine the quantity of analyte in the sample.

### Amplification Reaction Heterogeneous Format II

An appropriate volume of sample to be tested and an appropriate volume of reagent containing primary binder and, for example, a magnetic particle solid phase coated with anti-analyte are dispensed into a chamber and incubated for an appropriate period of time. The primary binder will bind to AUA and the complex will be captured by the magnetic particle solid phase. The particles are magnetically separated and washed. Amplifier is added. The amplification reaction will be initiated by the CE exposed by the primary binder bound to analyte. An aggregate of amplifier molecules can form on the solid phase. After washing, a labeled binder specific for the aggregate is added, incubated for an appropriate period of time, washed and detected. The binder can be labeled with any signal generating molecule, for example a fluorescent molecule for detection. This format produces a standard forward sandwich immunoassay with detection integrating the entire bulk solution. This format is not a single molecule detection technology but requires a standard curve to determine the quantity of analyte in the sample.

### Amplification Reaction Heterogeneous Format III

An appropriate volume of sample and an appropriate volume of reagent containing primary binder and solid phase coated with anti-analyte are combined and incubated for an appropriate period of time. The primary binder binds the analyte and the complex is captured on the solid phase. After an appropriate incubation time the supernatant is removed and the solid phase washed. Labeled amplifier is added and incubated for an appropriate period of time, the supernatant removed, washed and detected.

### Amplification Reaction Histopathology

The technology can also be formatted to identify the presence of a biomarker in a tissue sample. For example, a fresh, frozen or embedded tissue sample is sectioned and incubated for an appropriate period of time with a primary binder and amplification reagents to form aggregates. The tissue sample is then placed directly under a microscope or washed and placed under a microscope to detect or quantify aggregates.

### Amplification Reaction Flow Cytometry

An appropriate volume of sample and an appropriate volume of primary binder and amplifier are combined. After an appropriate period of time the sample is passed through the flow cytometry detection system.

### FRET Chamber Format

One embodiment of the present technology dispenses an appropriate volume of sample to be tested and a volume of reagent containing primary binder and a labeled D/A pair into a chamber and the mixture incubated for an appropriate period of time. The resulting aggregates are read.

### FRET Filter Format

The FRET can be used in the filter format. In this case, the CUA or EUA is incubated with primary binder and a FRET pair for an appropriate period of time. The reaction product is captured on a filter, washed and read. (See discussion regarding the Amplification Filter Format above for details about the typical filters that can be used.)

### FRET Heterogeneous Format I

The FRET can be formatted as a classical heterogeneous immunoassay. In this case the AUA, CUA or EUA is captured onto a solid phase and washed if appropriate. Next, a primary binder and FRET pair are added and incubated for an appropriate period of time. The sample is washed if appropriate and then read.

### FRET Histopathology Format

The technology can also be formatted to identify the presence of a biomarker in a tissue sample. For example, a fresh, frozen or embedded tissue sample is sectioned and incubated for an appropriate period of time with a primary binder and FRET pair. The tissue sample is then placed directly under a microscope or washed and placed under a microscope to detect or quantify signal generating clusters.

### FRET Flow Cytometry Format

An appropriate volume of sample and an appropriate volume of primary binder and a FRET pair are combined. After an appropriate period of time the sample is passed through the flow cytometry detection system.

### POC Instrumentation

The technology can be formatted to accommodate single samples read in a hand-held, battery-operated reader.

### Central Laboratory Instrumentation

The technology can also be formatted for high throughput applications using a random access linear processor capable of processing >50 samples per hour.

### Qualitative Format

The technology can also be formatted for low sensitivity applications using a visual semi-quantitative read out. Aggregates are visually detected by light scattering. The process is made semi-quantitative by comparison to standards.

### Therapeutic Format

The technology can also be formatted for therapeutic purposes. For example, the primary binder may be specific for a marker expressed on a malignant cell and the amplifier or amplifiers labeled with a cytotoxic or radioactive label.

The technology may also be used for in vivo imaging.

### Viral Analysis Using Conformational Epitopes

It should be noted that viral identification creates a unique set of conditions for the use of conformational epitope initiate signal generation. Most eukaryotic and prokaryotic cells range in size between 1 and 10 um in diameter and display thousands of copies of antigen on their surface. This level of antigen expression is sufficient to identify antibody mediated signal generation and is commonly used in IHC, flow cytometry and in microscopy to identify pathogens. Viruses, however, are much smaller. Most human pathogens have a diameter of 100 nm +/−50 nm and display only hundreds of copies of surface antigens. Viral particles, therefore, do not generate enough signal to be identified by direct light microscopy. In addition, the virus may be present at low concentration and therefore must be identified in a large sample volume. The virus may also be coated with host antibodies further reducing the number of available binding sites for identification or quantification.

### Viral Detection

One embodiment of the technology uses the amplification format or the FRET linear amplification format to detect virus from large sample volume. An exemplary reaction includes:
Sample—serum, plasma, blood, urine, respiratory specimen, CSF.
Sample volume >0.1 ml (˜0.1-10 ml).
Add mouse antibody specific for EUA.
Add magnetic particles coated with a mix of human and mouse anti- FcgR1 binding site antibodies, anti-C1Q binding site antibodies or both and incubate for an appropriate period of time.
Magnetically separate and remove unbound material.
Suspend the magnetic particles and disrupt bound complexes with pH or detergent or both.
Separate magnetic particle.
Transfer an appropriate volume of supernatant to a detection chamber, add primary binder and amplifiers or FRET linear amplification reagents, incubate for an appropriate period of time and read or filter and read.

Some advantages of using linear amplification format to detect viruses in large samples includes, direct identification of virus particles which is not possible by immunological methods, large volume interrogation, sensitive, rapid and easy to use.

### Conformational Epitopes as a Method of Sample Preparation—Isolation and Concentration of the EUA

One of the biggest challenges of high sensitivity assays is sample preparation. Samples may need processing to be accommodated by the assay format. These processing steps add cost, complexity and are inconvenient. Reasons for sample processing include:

The AUA or EUA is present at very low concentration in a large volume of sample. The volume must be reduced to fit assay format and instrument detection system design.

Interfering substances may be present and need to be removed to obtain accurate results.

The AUA or EUA may exist as a complex or structure that makes it inaccessible.

One convenient and commonly employed method to achieve sample concentration or remove inhibitors is to capture the AUA or EUA with specific antibodies immobilized on a solid phase support which is then separated from the starting material. Examples include magnetic particles and plastic beads. This approach works well for many applications but may be limited when dealing with samples derived from blood, urine, respiratory or nasopharyneal origin. The AUA or EUA frequently is covered by or found in complex with antibodies from the patient's natural immune response making binding with AUA or EUA with exogenously supplied specific antibodies impossible or improbable. Furthermore, it is impractical to try to isolate these complexes from blood, serum or plasma with a binder specific for a stable epitope on the antibody in complex because the circulating concentration of immunoglobulin approaches 15 mg/ml in a healthy individual. The use of conformational epitopes, however, can be used as a practical solution to the problem.

For example, human anti-C1Q binding site binders and/or anti-FcgR1 binding site binders can be immobilized on the surface of a MP. Coated MPs are added to the sample and incubated for an appropriate period of time. Immune complexes are captured by the MPs. After magnetic separation, the complexes are disrupted by heat, acid or detergent before entry into a detection format.

For example, a primary binder to any conformational epitope on a human antibody is immobilized on a magnetic particle and incubated with sample. After an appropriate period of time the magnetic particles are separated, unbound material removed and the bound complexes are disrupted with heat, pH change or detergent and further processed as required.

It is possible that samples will run the entire range from no immune response to saturating levels of antibody. An EUA may have none or all of its antigens in complex. To assure detection of the EUA in all cases, a mouse anti-EUA PB can be added to the sample along with solid phase coated with a mixture of human and mouse anti-C1Q binding site binder or human and mouse anti-FcgR1 binding site binder, or a combination thereof (FIG. 13).

FIG. 13 diagrams the configuration of reagents designed to utilize conformational epitopes to isolate, for example, proteins, cells or viruses from complex mixtures. In this specific example, a virus 1 presents an antigen 2 that is bound by an antibody 3 that produces the C1Q binding site 65 and the FcgR1 binding sites 66. Antibodies with specificity for the C1Q binding site 71 and the FcgR1 binding site 72 are immobilized on the magnetic particle 73. To a measured volume of sample (serum, plasma or blood) a mouse antibody specific for the virus under assay is added to the sample along with magnetic particles coated with a mixture of mouse anti-C1Q binding site and anti-FcgR1 binding site antibodies and human anti-C1Q binding site and anti-FcgR1 binding site antibodies. If the virus is coated with human immune complexes the magnetic particles coated with human anti-C1Q and anti-FcgR1 binding site binders will capture the complexes. If there is no immune response, or the virus is not completely covered with human antibodies, the mouse antibodies will react with the virus and magnetic particles coated with mouse anti-C1Q binding site and mouse anti-FcgR1 binding site binders will capture the complexes. After an appropriate incubation period, the sample is subjected to a magnetic field and unbound material discarded. The sample is then further processed to identify the presence of specific pathogens.

Conformational Epitopes as a Method for Protein Isolation from Complex Mixes

The reagents developed above for the present technology can be used to isolate and concentrate proteins produced by cell cultures. A continuous process is possible with the following design:
protein secreted into culture supernatant
binding to specific Ab in supernatant
circulate over a bead bed with immobilized binder or binders to conformational epitopes on the specific antibody
remove bead bed
dissociate Ag-Ab complexes from bead bed
return bead bed to system
dissociate Ag-Ab complex
return Ab to system
process secreted protein as required.

### Method of Multiplexing

Embodiments of the present technology can be formatted so that multiple primary binders with multiple specificities are brought in contact with a sample in a single reaction tube. Each primary binder would have the ability to trigger the amplification reaction if its specific partner is present in the sample. Using this format, multiple analytes can be under assay at the same time from a single sample or a pool of samples in a single tube. Alternatively, amplifiers labeled with different fluorophores that are specific for each primary binder can be used with different filters and/or excitation wavelengths in the detection system. This may be the only technology that will permit the simultaneous interrogation of an antigen and a nucleic acid in the same tube at the same time.

Embodiments of the present technology can be formatted so that multiple primary binders with multiple specificities are brought in contact with a sample in a single reaction tube. Each primary binder would have the ability to trigger the FRET reaction if its specific partner is present in the sample. Using this format, multiple analytes can be under assay at the same time from a single sample or a pool of samples in a single tube. Alternatively, FRET pairs with different D/A pairs that are specific for each primary binder can be used with different filters and/or excitation wavelengths in the detection system.

General Methods used in carrying out the above processes are known to one skilled in the art and include, but are not limited to the following: molecular cloning, assay development, conjugation methods, labeling of molecules, biotinylation of molecules, and the like. These techniques are known to those with experience in this field, and descriptions of the techniques can be found in general references, such as:
Immunoassays A Practical Guide, Brian Law, Taylor and Francis, 1996
Immunoassays A Practical Approach, James P. Gosling, Oxford University Press, 2000
Molecular Cloning: A Laboratory Manual (Third Edition), Joseph Sambrook, David Russel, Cold Spring Harbor Laboratory Press, 2001
PCR Cloning Protocols (2^{nd} Edition), Bing-Yuan Chen, Harry W. Jones, Humana Press, 2002
Sigma Aldrich, On line Technical Documents, Search—Conjugation Reagents
Chattopadhaya et al., “Strategies for site-specific protein biotinylation using in vitro, in vivo and cell free systems: toward functional protein arrays,” Nature Protocols 1/5 (2006) 2386-2398.

The following examples are intended to illustrate the invention, but not limit its scope.

### EXAMPLES

Example 1

A stock solution of Troponin-T, 10pg/ml, is prepared in phosphate-buffered saline containing 2 mg/ml bovine serum albumin and 1 mg/ml mouse IgG. In separate tubes, 1 ul, 10 ul and 100 ul of the Troponin-T stock solution is added to 99 ul, 90 ul or 0 ul of the phosphate buffer above respectively. 100 ul of a reagent containing mouse antibody to Troponin-T at 100 nM and two amplifier binders, one specific for the mouse antibody C1Q binding site and one specific for the mouse antibody FcgR1 binding site, each present at 20 uM in phosphate-buffered saline containing 2 mg/ml bovine serum albumin and 1 mg/ml mouse IgG is added to each tube. The reaction is incubated for 15 minutes and read in a chamber of dimension 3.2 cm×3.2 cm×0.2 mm and imaged onto a 20 mpix CCD. The amplifier binders are labeled with fluorescein. The output is shown as a series of spots on the detector when each tube is analyzed, showing the presence of Troponin-T in the tubes and confirming the utility of the amplification assay for Troponin-T over the concentration range tested.

### Example 2

A stock solution of Troponin-T, 1 pg/ml, is prepared in pooled human serum. In separate tubes, 1 ul, 10 ul and 100 ul of the Troponin-T stock solution is added to 99 ul, 90 ul or 0 ul of pooled human serum. 100 ul of a reagent containing mouse antibody to Troponin-T at 100 nM and two amplifier binders, one specific for the mouse antibody C1Q binding site and one specific for the mouse antibody FcgR1 binding site, each present at 20 uM in phosphate-buffered saline containing 2 mg/ml bovine serum albumin and 1 mg/ml mouse IgG is added to each tube. The reaction is incubated for 15 minutes and read in a chamber of dimension 3.2 cm×3.2 cm×0.2 mm and imaged onto a 20 mpix CCD. The amplifier binders are labeled with fluorescein. The output is shown as a series of spots on the detector when each tube is analyzed, showing the presence of Troponin-T in the tubes and confirming the utility of the amplification assay for Troponin-T in human serum over the concentration range tested.

### Example 3

A metastatic breast cancer cell line expressing Epithelial Cell Adhesion Molecule, EpCAM, is obtained from the American Type Culture Collection. The cell line is propagated and cells harvested and quantified. A mouse anti-EpCAM antibody is obtained from Santa Cruz Biotechnology. Mouse anti-C1Q binding site and mouse anti-FcgR1 binding site Fabs are isolated from a phage display library, Creative Biolabs. Cells are spiked into 3 separate tubes containing phosphate buffered saline containing 1 mg/ml bovine serum albumin and 1 mg/ml mouse IgG to give 100, 1000, and 10,000 cells in a final volume of 100 ul respectively. 100 ul of a reagent containing 100 nM mouse anti-EpCAM antibody and 4.2E-9M anti-C1Q binding site and mouse anti-FcgR1 binding site Fabs labeled with Alexa 594/Alexa610 respectively in phosphate buffered saline containing 1 mg/ml bovine serum albumin and 1 mg/ml mouse IgG is added to each tube. The reaction is incubated for 60 minutes and read in a 3.2 cm×3.2 cm×0.2 mm chamber imaged onto a 20 mpix CCD. The output is shown as a series of spots on the detector when each tube is analyzed, confirming the utility of the FRET assay for the EpCAM over the concentration range tested.

### Example 4

An experiment is conducted to determine the presence of Analyte A, using the amplifier method described herein. The concentration of the amplifier binder is 1-100 uM, the association rate constant is 2-10 E5(M-1s-1), and the reaction is conducted in an instrument having a chamber of specific dimensions and a charge coupled device having 12-30 megapixels. The chamber has dimensions 3.2 cm×3.2 cm, where the depth of solution does not exceed 0.3 mm. The output is shown as spots on the detector, showing the presence of Analyte A, confirming the utility of the amplification assay for Analyte A.

### Example 5

An experiment is conducted to determine the quantity of Analyte A, using the FRET method described herein. The exposed epitopes on the primary binder are within 10 nm of each other. The concentration of each of the donor and receptor molecules is between 3.7E-11 and 2.8E-7M, the association rate constant is 2-10 E5 (M-1s-1), and the reaction is conducted in an instrument having a chamber with dimensions 3.2 cm×3.2 cm, where the depth of solution does not exceed 0.3 mm. The instrument has a charge coupled device having 12-30 megapixels. Calibrations with known quantities of Analyte A that bracket the expected concentration of Analyte A in the test sample are run. An increasing number of spots is detected as the concentration of analyte A is increased. The output of the test sample is shown as spots having intensity on the detector. The number of spots determines the quantity of Analyte A in the sample. The system calibrators assure a properly operating system. This experiment demonstrates the utility of the FRET method for determination of the quantity of Analyte A.

### Example 6

An experiment is conducted to determine the presence of Analyte A, using the amplifier method described herein. The concentration of the amplifier binder is 0.05-100 uM, the association rate constant is 0.1-10 E5(M-1s-1), and the reaction is conducted in an instrument having a chamber with dimensions between 4 cm×4 cm to 1 cm×5 cm where the depth of solution is between 0.01 and 1 mm and a charge coupled device having 5-30 megapixels. The output is shown as spots on the detector, showing the presence of Analyte A, confirming the utility of the amplification assay for Analyte A.

### Example 7

An experiment is conducted to determine the quantity of Analyte A, using the FRET method described herein. The exposed epitopes on the primary binder are within 10 nm of each other. The concentration of each of the donor and receptor molecules is between 4.6E-12 and 2.8E-6, the association rate constant is 0.1-10 E5 (M-1s-1), and the reaction is conducted in an instrument having a chamber with dimensions between 4 cm×4 cm to 1 cm×5 cm, where the depth of solution is between 0.01 and 1 mm. The instrument has a charge coupled device having 5-30 megapixels. Calibrations with known quantities of Analyte A that bracket the expected concentration of Analyte A in the test sample are run. An increasing number of spots is detected as the concentration of analyte A is increased. The output of the test sample is shown as spots having intensity on the detector. The number of spots determines the quantity of Analyte A in the sample. The system calibrators assure a properly operating system. This experiment demonstrates the utility of the FRET method for determination of the quantity of Analyte A.

Those with ordinary skill in this technology area will recognize that variations of the above disclosure are contemplated to be within the scope of the invention. The present technology is now described in such full, clear and concise terms as to enable a person skilled in the art to which it pertains, to practice the same. It is to be understood that the foregoing describes preferred embodiments of the present technology and that modifications may be made therein without departing from the spirit or scope of the present technology as set forth in the appended claims. Further the examples are provided to not be exhaustive but illustrative of several embodiments that fall within the scope of the claims.

### LITERATURE

1. Jefferis, “Antibody Therapeutics: Isotype and Glycoform Selection,” Expert opinion on biological therapy 7/9 (2007) 1401-1413.
2. Carter, “Potent antibody therapeutics by design,” Nature Reviews Immunology 6 (2006) 343-357.
3. Holliger et al., “Engineered antibody fragments and the rise of single domains,” Nature Biotechnology 23/9 (2005) 1126-1136.
4. Skerra, “Alternative non-antibody scaffolds for molecular recognition,” Current Opinion in Biotechnology 18 (2007) 295-304.
5. Maynard et al., “Antibody Engineering,” Annual Reviews of Biomedical Engineering 2 (2000) 339-376.
6. Eisenhardt et al., “Subtractive Single-Chain Antibody (scFv) Phage—

Display: tailoring phage display for high specificity against function-specific conformations of cell membrane molecules,” Nature Protocols 2/12 (2007) 3063-3073.
7. Almagro et al., “Humanization of Antibodies,” Frontiers in Bioscience 13 (2008) 1619-1633.
8. Kim et al., “Antibody Engineering for the Development of Therapeutic Antibodies,” Molecules and Cells 20/1 (2005) 17-29.
9. Wilson et al., “Antibody-antigen interactions: new structures and new conformational changes,” Current Opinion in Structural Biology 4 (1994) 857-867.
10. Webster et al., “Antibody-antigen interactions,” Current Opinion in Structural Biology 4 (1994) 123-129.
11. Lippow et al., “Computational design of antibody affinity improvements beyond in vivo maturation,” Nature Biotechnology 25 / 10 (2007) 1171-1176.
12. Glaser et al., “Novel Antibody Hinge Regions for Efficient Production of CH2 Domain- deleted Antibodies,” The Journal of Biological Chemistry 280/50 (2005) 41494-41503
13. Swers et al., “Shuffled Antibody Libraries Created By In Vivo Homologous Recombination and Yeast Surface Display,” Nucleic Acids Research 32/3 (2004) e36.
14. Patrick et al., “In Vitro Selection and Characterization of a Stable Domain of Phosphoribosylanthranilate Isomerase,” FEBS Journal 272 (2005) 3684-3697.
15. Rodi et al., “Phage Display in Pharmaceutical Biotechnology,” Current Opinion in Biotechnology 10 (1999) 87-93.
16. Sidhu., “Phage Display in Pharmaceutical Biotechnology,” Current Opinion Biotechnology 11 (2000) 610-616
17. Idusogie et al., “Mapping of the C1Q Binding Site on Rituxan, a Chimeric Antibody with a Human IgG1 Fc,” The Journal of Immunology 164 (2000) 4178-4184.
18. Gadjeva et al., “Interaction of Human C1q with IgG and IgM: Revisited,” Biochemistry (2008) 47 13093-13102.
19. Carters et al., “Design and Use of Scorpions Fluorescent Signaling Molecules,” Methods Molecular Biology 429 (2008) 99-115.

1. A method for determining the presence or quantity of analyte molecules or entities in a sample, comprising
a. reacting each unit of sample with a primary binder, the primary binder having specificity for the analyte, to form an analyte-primary binder complex, wherein the primary binder comprises one or more hidden epitopes, wherein the hidden epitopes of the primary binder become exposed upon the primary binder binding to the analyte,
b. reacting the analyte-primary binder complex with a signal generating system, wherein the signal generating system binds to the exposed epitopes of the primary binder forming a cluster of analyte-primary binder-signal generating molecules, wherein said cluster resides in a thin chamber or on the surface of a porous filter and a signal from the cluster is imaged onto a CCD, and
c. analyzing the presence or quantity of said cluster signal as a means of determining the presence or quantity of the analyte molecule or entity.

2. The method of claim 1 for determining the presence or quantity of analyte molecules or entities in a sample, wherein the analyte is selected from the group consisting of antigens, proteins, nucleic acids, lipids, carbohydrates, steroids, cells, viruses and other informative biomarkers.

3. The method of claim 2 for determining the presence or quantity of analyte molecules or entities in a sample, wherein the analyte is a protein.

4. The method of claim 1 for determining the presence or quantity of analyte molecules or entities in a sample, wherein the analyte-primary binder-signal generating system complex also optionally comprises a an amplifier linker or a FRET linker.

5. The method of claim 1 for determining the presence or quantity of analyte molecules or entities in a sample,
wherein the signal generating system comprises at least one labeled amplifier binder, wherein the signal generating system comprises reacting said analyte—primary binder complex with the at least one labeled amplifier binder to form an analyte—primary binder—labeled amplifier binder complex,
wherein the labeled amplifier binder is capable of reacting with the exposed epitopes of the analyte-primary binder complex,
wherein each of the labeled amplifier binders comprises more than one hidden conformational epitopes, wherein the hidden conformational epitopes of the labeled amplifier binders become exposed when bound to the analyte-primary binder complex,
and wherein additional labeled amplifier binder molecules continue to assemble in a continuous process by binding exposed epitopes displayed by previously bound labeled amplifier molecules forming a cluster an aggrcgatc of labeled molecules that produces a signal above background with time.

6. The method of claim 4 for determining the presence or quantity of analyte molecules or entities in a sample,
wherein the signal generating system comprises at least one labeled amplifier binder, wherein the signal generating system comprises reacting said analyte—primary binder—amplifier linker—complex with the at least one labeled amplifier binder to form an analyte—primary binder—amplifier linker—labeled amplifier binder complex,
wherein the labeled amplifier binder is capable of reacting with the exposed epitopes of the analyte-primary binder complex,
wherein each of the labeled amplifier binders comprises more than one hidden conformational epitopes, wherein the hidden conformational epitopes of the labeled amplifier binders become exposed when the labeled amplifier binder is bound to the analyte-primary binder—amplifier linker complex,
and wherein additional labeled amplifier binder molecules continue to assemble in a continuous process by binding exposed epitopes displayed by previously bound labeled amplifier molecules forming a cluster of labeled molecules that produces a signal above background with time.

7. The method of claim 6 for determining the presence or quantity of analyte molecules or entities in a sample, wherein the analyte is a protein and wherein the primary binder and the one or more labeled amplifier binders and amplifier linker are selected from the group consisting of antibodies, antibody binding fragments, engineered antibody binders, and other protein binders.

8. The method of claim 6 for determining the presence or quantity of analyte molecules or entities in a sample, wherein the analyte and primary binder are both nucleic acids and the one or more labeled amplifier binders and amplifier linker are selected from the group consisting of antibodies, antibody binding fragments, engineered antibody binders, and other protein binders.

9. The method of claim 6 for determining the presence or quantity of analyte molecules or entities in a sample, wherein the exposed conformational epitopes of the labeled amplifier binders are the same.

10. The method of claim 6 for determining the presence or quantity of analyte molecules or entities in a sample, wherein the exposed lidden conformational epitopes of the labeled amplifier binders are not the same.

11. The method of claim 6 for determining the presence or quantity of analyte molecules or entities in a sample, wherein the concentration of the amplifier binders is 0.05-100 uM, the association rate constant is 0.1-10 E5 (M-1s-1), and the reaction is conducted in an instrument having a chamber of specific with a depth between 0.01 and 1 mm and a charge coupled device having 5-30 megapixels.

12. The method of claim 11 wherein said amplifier binders bind at or near the C1Q binding site and FcgR1 binding site.

13. The method of claim 11 for determining the presence or quantity of analyte molecules or entities in a sample, wherein the chamber has dimensions 4 cm×4 cm to 1 cm×5 cm, where the depth of solution is between 0.01 and 1 mm.

14. The method of claim 13 for determining the presence or quantity of analyte molecules or entities in a sample, wherein said assay is conducted by collecting the analyte-primary binder-signal generating system complex on a porous filter, wherein the porous filter has a surface dimension between 0.5x0.5 and 3.2x3.2 cm in a device having a depth between 0.5 and 4 cm, and wherein the volume of solution containing the complex is between 0.1 and 10 ml.

15. The method of claim 6 for determining the presence or quantity of analyte molecules or entities in a sample, wherein the concentration of the amplifier binders is about 1−about 100 uM, the association rate constant is about 2-10 E5 (M-1s-1), and the reaction is conducted in an instrument having a chamber of with a depth between 0.1 and 0.3 mm and a charge coupled device having 12-30 megapixels.

16. The method of claim 15, wherein said amplifier binders bind at or near the Cl Q binding site and FcgR1 binding site.

17. The method of claim 15 for determining the presence or quantity of analyte molecules or entities in a sample, wherein the chamber has dimensions of 3.2 cm×3.2 cm, where the depth of solution is between 0.1 and 0.3 mm.

18. The method of claim 15 for determining the presence or quantity of analyte molecules or entities in a sample, wherein the assay is conducted by collecting said analyte-primary binder-signal generating system complex on a porous filter, wherein the porous filter has a surface dimension between 0.5x0.5 and 3.2x3.2 cm in a device having a depth between 0.5 and 4 cm, and wherein the volume of solution containing the complex is between 0.1 and 10 ml.

19. The method of claim 1 for determining the presence or quantity of analyte entities in a sample displaying multiple primary binder binding sites, wherein said primary binder has two or more different epitopes, at least one of the epitopes being hidden until the primary binder binds to the analyte and being exposed after the primary binder binds to said analyte, and
wherein the exposed epitopes bind to a signal generating system comprising secondary binders specific for the exposed epitopes labeled with a donor and acceptor FRET pair, forming a cluster of primary binders with bound FRET pairs the signal from said cluster being created when the FRET pairs are energized.

20. The method of claim 4 for determining the presence or quantity of analyte entities in a sample displaying multiple primary binder binding sites, wherein said primary binder has two or more different epitopes, at least one of the epitopes being hidden until the primary binder binds to the analyte and being exposed after the primary binder binds to said analyte,
wherein said exposed epitope binds a FRET linker, wherein said FRET linker has two or more different epitopes, at least one of the epitopes being hidden until the FRET linker binds to the primary binder and being exposed after the FRET linker binds to said primary binder, and
wherein the FRET linker binds a signal generating system comprising FRET pairs with the exposed epitopes on the FRET linker secondary forming a cluster of FRET linkers with bound FRET pairs, the signal from said cluster being created when the FRET pairs are energized.

21. The method of claim 19 for determining the presence or quantity of analyte entities in a sample displaying multiple primary binder binding sites, wherein the analyte entities is a protein or cell displayed antigen and wherein the primary binder and the secondary binders labeled with a donor and acceptor FRET pair are selected from the group consisting of antibodies, antibody binding fragments, engineered antibody binders, and other protein binders.

22. (canceled)

23. The method of claim 20 for determining the presence or quantity of analyte entities in a sample displaying multiple primary binder binding sites, wherein the exposed epitopes of the primary binder or FRET linker are within 10 nm of each other, the concentration of each of the donor and acceptor secondary binder is between about 4.6E-12 and about 2.8E-6 M, the association rate constant for the donor and acceptor secondary binder is about 0.1 to about 10 E5 (M-1s-1), and the reaction is conducted in an instrument having a chamber with a depth between 0.01 and 1 mm of and a charge coupled device having 5-30 megapixels.

24. The method of claim 23 wherein said donor and acceptor secondary binders bind at or near the C1Q binding site and FcgR1 binding site.

25. The method of claim 23 for determining the presence or quantity of analyte entities in a sample displaying multiple primary binder binding sites, wherein the chamber has dimensions 4 cm×4 cm to 1 cm×5 cm, where the depth of solution is between 0.01 and 1 mm.

26. The method of claim 23 for determining the presence or quantity of analyte entities in a sample displaying multiple primary binder binding sites, wherein the analyte-primary binder-signal generating system complex is collected on a porous filter,
wherein said porous filter has a surface dimension between 0.5x0.5 and 3.2x3.2 cm in a device having a depth between 0.5 and 4 cm, and wherein the volume of solution containing said complex is between about 0.1 and about 10 ml.

27. The method of claim 20 for determining the presence or quantity of analyte entities in a sample displaying multiple primary binder binding sites, wherein the exposed epitopes of the primary binder or FRET linker are within 10 nm of each other, the concentration of each of the donor and acceptor secondary binder is between 3.7E-11 and 2.8E-7M , the association rate constant for the donor and acceptor secondary binder is about 2-10 E5 (M-1s-1), and the reaction is conducted in an instrument having a chamber having a depth between 0.1 and 0.3 mm and a charge coupled device having 12-30 megapixels.

28. The method of claim 27 wherein said donor and acceptor secondary binders bind at or near the C1Q binding site and FcgR1 binding site.

29. The method of claim 27 for determining the presence or quantity of analyte entities in a sample displaying multiple primary binder binding sites, wherein the chamber has dimensions of 3.2 cm×3.2 cm, where the depth of solution is between about 0.1 and about 0.3 mm.

30. The method of claim 27 for determining the presence or quantity of an analyte entities in a sample displaying multiple primary binder binding sites, wherein the analyte-primary binder-signal generating system complex is collected on a porous filter,
wherein the porous filter has a surface dimension between 0.5x0.5 and 3.2x3.2 cm in a device having a depth between 0.5 and 4 cm, and wherein the volume of solution containing the complex is between about 0.1 and about 10 ml.

31. A method for rapid determination of the presence or quantity of analyte molecules or entities in a sample, comprising
a. reacting each unit of sample with a primary binder, the primary binder having specificity for the analyte, to form an analyte-primary binder complex, wherein the primary binder, upon binding the single analyte molecule, exposes binding sites for secondary binders, wherein said secondary binders carry a signal generating molecule,
b. reacting the analyte-primary binder complex with the secondary binders to form a signal generating cluster, wherein said cluster resides in a chamber or on the surface of a porous filter and a signal from the cluster is imaged onto a CCD,
c. analyzing the presence or quantity of said cluster signal as a means of determining the presence or quantity of the analyte molecule or entity.

32. The method of claim 31 for rapid determination of the presence or quantity of the analyte molecules or entities in a sample,
wherein said secondary binder comprises at least one labeled amplifier binder,
wherein the labeled amplifier binder is capable of reacting with the exposed epitopes of the analyte-primary binder complex,
wherein each of the labeled amplifier binders comprises more than one hidden conformational epitopes, wherein the hidden conformational epitopes of the labeled amplifier binders become exposed when bound to the analyte-primary binder complex,
and wherein additional labeled amplifier binder molecules continue to assemble in a continuous process by binding exposed epitopes displayed by previously bound labeled amplifier molecules, forming a cluster of labeled molecules that produces a signal above background with time.

33. The method of claim 31 for rapid determination of the presence or quantity of analyte entities displaying multiple primary binder binding sites in a sample,
wherein said primary binder has two or more different epitopes, at least one of the epitopes being hidden until the primary binder binds to the analyte and being exposed after the primary binder binds to said analyte, and
wherein the exposed epitopes bind a signal generating system comprising secondary binders specific for the exposed epitopes, wherein said secondary binders are labeled with a donor and acceptor FRET pair, forming a signal generating cluster, wherein said cluster resides in a thin chamber or on the surface of a porous filter and a signal from the cluster is imaged onto a CCD, and
analyzing the presence or quantity of said cluster signal as a means of determining the presence or quantity of the analyte entities.

34. The method of claim 32 for rapid determination of the presence or quantity of the analyte molecules or entities in a sample, wherein said determination is completed within approximately 2-15 minutes.

35. The method of claim 33 for rapid determination of the presence or quantity of analyte entities in a sample displaying multiple primary binder binding sites, wherein said determination is completed within approximately 2-44 minutes.
